(19) United States
(12) Patent Application Publication
(10) Pub. No.: US 2011/0082081 A1
(43) Pub. Date: Apr. 7, 2011

(54) COMPOSITIONS AND METHODS FOR REPAIR OF TISSUES

(75) Inventors: Daniel D. Carson, Houston, TX (US); Mary C. Farach-Carson, Houston, TX (US); Weidong (William) Yang, Newark, DE (US)

(73) Assignee: University of Delaware, Newark, DE (US)

(21) Appl. No.: 12/902,723

(22) Filed: Oct. 12, 2010

Related U.S. Application Data
(62) Division of application No. 11/811,378, filed on Jun. 7, 2007.
(60) Provisional application No. 60/813,537, filed on Jun. 14, 2006.

(51) Int. Cl. A61K 38/18; A61P 43/00; A61P 19/02

(52) U.S. Cl. 514/8.8; 514/7.6

(57) ABSTRACT

Biomaterials providing sustained release of growth factor for repair of tissues such as bone and cartilage are disclosed. The biomaterials comprise a proteoglycan derived from domain I of perlecan and a growth factor, and, optionally, collagen.

### RELATED APPLICATIONS

This application claims priority to U.S. 60/813,537, filed on Jun. 14, 2006 which is incorporated herein by reference.

### GOVERNMENT INTERESTS

The Government may have certain rights in this invention under Grant Nos. R01-DE13542 and P20-PR16458 and National Research Service Award F32-AG20078 awarded by the National Institutes of Health.

### FIELD OF THE INVENTION

This invention relates to the field of tissue repair. Specifically, the invention relates to in situ mammalian tissue repair.

### BACKGROUND OF THE INVENTION

The proper treatment and healing of damaged tissues is a challenge. Improper healing can lead to life long complications. Protracted healing times are also a concern due to the costs of treatment and extended potential for complications. The healing of cartilaginous tissues, which includes without limitation, meniscus and cartilage, and the healing of related ligament, tendon, bone, skin, cornea and periodontal tissues, is especially challenging because a lack of tissue vascularization slows the healing process. Devices and methods to accelerate cartilaginous tissue regeneration are highly desired to minimize healing time and promote proper healing of cartilaginous tissues.

Cartilage is an avascular deformable tissue consisting of sparsely embedded chondrocytes in a specialized extracellular matrix (ECM). The avascular aspect of cartilage inhibits the appearance of inflammatory and pluri-potential repair cells. This ECM has dense collagen and proteoglycan networks that determine mechanical and functional properties of the tissue (1-3). The ECM imprisons resident chondrocytes in a matrix non-conducive to migration. Thus the natural response to repair in adult articular cartilage is a weak response or no repair response.

The primary collagen component in cartilage is collagen II that interacts with the quantitatively minor collagens IX and XI to form heterotypic fibrils (1, 2). Proteoglycan interactions with collagen fibrils and growth factors have been implicated in the regulation of ECM assembly and growth factor functions (2-4). Perlecan (Pln) is a heparan sulfate proteoglycan (HSPG) with a protein core of approximately 400 kDa and consists of five distinct domains (5). Pln domain I (PlnDI) is a 22 kDa protein core that contains three ser-asp-gly (SDG) motifs that serve as glycosaminoglycan (GAG) attachment sites decorated with two to three heparan sulfate (HS) chains and one chondroitin sulfate (CS) chain (5-8) of heterogeneous size. Through GAG chains attached to PlnDI, Pln functions as a ligand reservoir for storage and protection of heparin-binding growth factors (HBGFs) including fibroblast growth factor-2 (FGF-2) (7, 8), vascular endothelial growth factor (VEGF) (9) and transforming growth factor β/bone morphogenetic proteins (TGF-β/BMPs) (6, 10, 11). Binding to GAG chains enhances the biological activities of these HBGFs (6, 7, 9-11). Thus, Pln and its GAG chains have a wide range of biological functions in cellular growth (7, 8), angiogenesis (9), development (3, 4, 6, 12) and tissue regeneration (13).

During skeletal development, Pln is found in cartilage anlagen after the expression of collagen II and aggrecan and is maintained as the major HSPG of adult cartilage (4, 6, 14, 15). Pln null mice exhibit disorganized growth plates, severe cartilage defects, and skeletal abnormalities (16-18). Several studies have demonstrated that Pln is crucial in chondrogenesis (3, 4, 6, 14, 19). These actions may occur in concert with growth factors (4, 9, 11), such as BMP-2 and TGF-β1 (6, 20, 21), or growth factor binding proteins, such as the BMP binding polypeptide, noggin (6, 22). As disclosed in US Patent Application Publication US 2004/0063619, this action of Pln can be useful in delivery systems for heparin-binding growth factors. In addition, Pln can maintain cartilage integrity and protect cartilage ECM from degradation (2, 17). The murine mesenchymal stem cell line, C3H10T1/2, plated on surfaces coated with either intact Pln or recombinant PlnDI attach and aggregate into dense cell condensations that express chondrogenic markers including collagen II, aggrecan and link protein (4, 14, 19, 20).

Collagen II fibrils support specific binding of a number of proteoglycans including fibromodulin (23, 24), biglycan (25) and aggrecan (25, 26). Both proteoglycan core proteins and their GAG chains mediate interactions with collagen II fibrils and modulate tensile strength of the ECM (25, 27-29). In addition to its biomechanical functions, collagen II also plays a role in induction of chondrogenesis (1, 3, 16, 30). Type IIA pro-collagen, but not type IIB collagen, binds BMP-2 and TGF-β1 (30). Other data suggest that interaction of BMP-2 with pro-collagen II is site-specific, and that the high affinity binding site is located in the D-period of the collagen triple helix (31). Based on these properties, collagen II has been used to prepare or modify scaffolds in cartilage engineering applications (32-36). Collagen II can support chondrocyte infiltration and attachment (32, 37, 38) and maintains chondrocyte morphology and phenotype (33, 34, 39, 40). Therefore, collagen II is an ideal candidate substrate to facilitate chondrogenesis and to use in cartilage tissue engineering.

During cartilage development, BMP-2 enhances recruitment of mesenchymal precursors to cartilage condensations, modulates expansion of condensation size and initiates BMP-dependent signaling cascades in mesenchymal progenitor cells for induction of chondrogenic differentiation (6, 41-43). Multi-potential precursor cells, such as C3H10T1/2 cells, cultured at high density initiate chondrogenesis following BMP-2 treatment (43-47). BMP-2 functions are enhanced by HS (4, 6, 10, 11). Also, collagen II can bind GAG chains attached to proteoglycans (27-29).

Current clinical treatments for symptomatic cartilage defects involve techniques aimed at: 1) removing surface irregularities by shaving and debridement; 2) penetration of subchondral bone by drilling, fracturing or abrasion to augment the natural repair response; 3) joint realignment or osteotomy to use remaining cartilage for articulation; 4) pharmacological modulation; 5) tissue transplantation; 6) cell transplantation; and 7) biomaterial mediated delivery and release of growth factors. Most of these methods have some short term benefit in reducing symptoms (months to a few years), while none have been able to consistently demonstrate successful repair in the long term.

Osteoarthritis, also known as degenerative arthritis or degenerative joint disease, is a condition in which low-grade inflammation results in pain in the joints, caused by wearing of the cartilage that covers and acts as a cushion inside joints. As the bone surfaces become less well protected by cartilage, the patient experiences pain upon weight bearing, including walking and standing. Due to decreased movement because of the pain, regional muscles may atrophy, and ligaments may become more lax. Treatment is often aimed at symptom relief. The 1995 American College of Rheumatology recommendations describe preliminary studies of disease-modifying osteoarthritis drugs (DMOADs), drugs whose action is not aimed principally at the control of symptoms, but instead at the prevention of structural damage in normal joints at risk for the development of osteoarthritis or to prevent the progression of structural damage in joints already affected by osteoarthritis. For the most part, approaches have been aimed at inhibiting the breakdown of articular cartilage by matrix metalloproteinases, or at stimulating repair activity by chondrocytes. A number of agents are under study, including matrix metalloproteinase inhibitors and growth factors. As of 1995, the American College of Rheumatology wrote that no agent had been shown to have a disease-modifying osteoarthritis effect in humans.

Several experimental techniques have been proposed to repair cartilage using growth factors alone or in combination with other biomaterials. A scaffold and/or hydrogel can be used along with species of soluble elements, e.g. heparin coated scaffolds (57). A major drawback of heparin coated scaffolds is that heparin has as an anti-coagulation effect on blood, thus hindering clotting and blood vessel repair at a wound site. Bone morphogenetic proteins have been combined with generic biomaterials such as polylactic acid (PLA), polyglycolic acid (PGA), collagen matrices and fibrin glues (Zhang et al. WO 00/44413), angiotensin-like peptides (Rodgers and Dizerega WO 00/02905), and extracts of bone containing a multiplicity of proteins called bone proteins (BP) (Atkinson, WO 00/48550). In the latter method, BMP soaked collagen sponges needed to be held in the cartilage defect using an additional fibrin/thrombin based adhesive, creating a rather complex and difficult to reproduce wound healing environment. Coating the biomaterial with fibronectin or RGD peptides to aid cell adhesion and cell migration has been done (Breckke and Coutts, U.S. Pat. No. 6,005,161). Some previous methods have combined bone-marrow stimulation with post-surgical injection of growth hormone in the synovial space with limited success (Dunn and Dunn, U.S. Pat. No. 5,368,051). Specific biomaterials compositions used to repair cartilage tissue damage include crushed cartilage and bone paste (Stone, U.S. Pat. No. 6,110,209), a multicomponent collagen-based construct (Pahcence et al., U.S. Pat. No. 6,080,194) and a curable chemically reactive methacrylate-based resin (Braden et al., U.S. Pat. No. 5,468,787).

Preferred would be a method that provides for sustained release of chondrogenesis growth factors at effective concentrations over prolonged periods of time. Such a sustained release would be advantageous over immediate release due to the longer healing time needed for avascular tissue repair relative to vascular tissue repair. Sustained release would also be greatly advantageous for the prevention of structural damage in joints at risk of developing osteoarthritis, as it could enhance or prevent decline of cartilaginous tissue over a prolonged period of time without requiring frequent dosaging.

Sustained-release formulations containing various polypeptide growth factors have been described. For example, WO 94/12158 describes growth hormone controlled-release systems formed by spraying a polymer and dry protein into a freezing solution of liquid nitrogen to form polymeric microspheres. U.S. Pat. No. 5,134,122 describes methods of forming microparticles that include salts of peptides such as LHRH. WO 96/37216 describes IGF-1 formulations comprising IGF-1 and hydrophobic polymers. EP 442,671 A2 describes microcapsules containing various polypeptides. Commonly a rate controlling synthetic bio-erodible polymer is used. Such systems are designed to release drug as the polymer erodes. This severely limits the selection of drug and polymer and can cause unintended immunological response complications.

### SUMMARY OF THE INVENTION

The present invention describes a biomaterial having immobilized thereon a proteoglycan-growth factor complex comprising (1) a proteoglycan that comprises an amino acid sequence of the core protein of domain I of a mammalian perlecan or that comprises an amino acid sequence having at least 90% homology to the core protein of domain I of a mammalian perlecan to which proteoglycan at least one glycosaminoglycan is attached and (2) at least one growth factor, said immobilized proteoglycan-growth factor complex being present in the biomaterial in a sufficient amount for sustained release of a therapeutically effective dose of growth factor to repair and regenerate tissue at a wound site over a predetermined period of time.

### BRIEF DESCRIPTION OF THE DRAWINGS

FIG. 1 (A-B) Area photograph of a representative dot blot depicting rhBMP-2 binding to PlnDI. FIG. 1(C) is a densiometric quantitation of these data.

FIG. 2 (A-B) Line graphs depict PlnDI-collagen II fibrils binding compared to a BSA control.

FIG. 3 (A-B) Bar graphs depict HS/CS biotinylated PlnDI binding to collagen II fibrils by measuring heparitinase and chondroitinase activity.

FIG. 4 Bar graph depicts comparison of biotinylated PlnDI binding to collagen II fibrils, collagen II monomers, heat denatured collagen II fibrils and BSA.

FIG. 5 Bar graph depicts comparison of BMP-2 binding to PlnDI-collagen II fibrils, heparitinase-digested PlnDI digested/collagen II fibril complexes, chondroitinase digested PlnDI/collagen II fibril complexes, collagen I-II and BSA.

FIG. 6 (A-B) Bar graphs depict BMP-2 release from PlnDI/collagen II fibril complexes and collagen II fibrils over time measured in days.

FIG. 7 Area photographs of high density micromass cultures of C3HT1/2 cells on PlnDI-collagen II fibril BMP-2, collagen II fibril-BMP-2, PlnDI-collagen II fibril and collagen II fibril substrates stained with Alcian blue.

FIG. 8 (A-C) Bar graphs of high density micromass cultures of C3H10T1/2 cells on PlnDI-collagen II fibril BMP-2, collagen II fibril-BMP-2, PlnDI-collagen II fibril and collagen II fibril substrates measuring chondrogenic differentiation in terms of detected levels of marker mRNA expression.

FIG. 9 (A) Area photograph depicts BMP-2 binding on the different scaffold substrates of PlnDI/collagen II fibrils-PLA, collagen II fibrils-PLA and PLA alone. FIG. 9 (B) Bar graph is a quantitation of the dye extracted from each scaffold indicating PlnDI binding effectiveness of the scaffold.

FIG. 10 (A-L) Area photographs of histological staining of C3H10T1/2 cells seeded and cultured for 21 day on scaffolds using various staining techniques. FIG. 10 (A) depicts a PlnDI/collagen II fibrils-PLA scaffold; FIG. 10 (B) depicts a collagen II fibrils-PLA scaffold; FIG. 10 (C) depicts a PLA scaffold; FIGS. 10 (D, G, J) depict a BMP-2-PlnDI/collagen II fibrils-PLA scaffolds; FIGS. 10 (E, H, K) depict a BMP-2-collagen II fibrils-PLA scaffolds; and FIGS. 10 (F, I, L) depict a BMP-2-PLA scaffolds.

FIG. 11 (A-L) Area photographs of immunohistochemical results of chondrogenic markers by C3H101/2 cells seeded on different scaffolds. FIGS. 11(A, D, G, J) depict a PlnDI/collagen II fibrils-BMP-2-PLA scaffolds; FIGS. 11(B, E, H, K) depict a collagen II fibrils-BMP-2-PLA scaffolds; FIGS. 11 (C, F, I, L) depict BMP-2-PLA scaffolds.

FIG. 12 (A-O) Area photographs of histological and immunohistochemical results of chondrogenic markers by mouse embryonic fibroblasts (MEFs) seeded on different scaffolds.

FIG. 13 Area photograph of a representative dot blot depicting PlnDI binding of FGF-2 vs. HEP-BSA binding of FGF-2 and BSA FGF-2 binding as a control.

### DETAILED DESCRIPTION OF THE INVENTION

The present invention concerns compositions for injection and devices for implantation in a mammalian body that facilitate sustained release of active agents comprising a growth factor useful in the repair and regeneration of tissues, especially cartilage, and methods of treating or preventing disorders of bone and cartilaginous tissue by administering such devices. It has been surprisingly found that when perlecan is bound to growth factors, perlecan provides for a sustained release of growth factors under physiological conditions.

In one embodiment, the invention provides a sustained release system that does not elicit an unintended immunological response and that harnesses the natural biological processes of avascular chondrogenesis to repair of tissues, and specifically cartilaginous tissues. In other embodiments, compositions and methods are provided that will facilitate in situ wound repair to accelerate the repair of tissues, especially cartilage.

One preferred embodiment of the invention uses a biomaterial to immobilize a proteoglycan-growth factor complex in which the growth factor is present in a sufficient amount to sustain delivery of a therapeutically effective dose of growth factor to repair and regenerate tissue at a wound site over a predetermined period of time. The biomaterial is preferably collagen or pro-collagen and most preferably collagen type II or pro-collagen type II-A. The growth factor is preferably a member of the heparin-binding growth factor family. In a preferred embodiment, a proteoglycan comprising perlecan domain I or an equivalent thereof having attached at least one glycosaminoglycan chain and at least one growth factor is bound to the biomaterial, preferably to collagen II fibrils that make up the biomaterial or are used to coat the biomaterial. The biomaterial can be injected and/or surgically implanted into a patient. The invention can be used to treat wound sites in skin, bone, or cartilaginous tissues and preferably bone or cartilaginous tissues.

In another embodiment, a composition of perlecan and growth factor is prepared and administered directly into a wound site, such as the synovial fluid of a knee or other joint, for repair or prevention of cartilage damage. Preferred perlecan molecules and growth factors are as discussed above.

### DEFINITIONS

The term “therapeutically effective amount” and similar terms used herein refers to an amount of a compound or combination of compounds that shows a pharmacological effect when administered in the mammalian body, such as ameliorates, attenuates or eliminates one or more symptoms of a particular disease or condition or prevents or delays the onset of one or more symptoms or a particular disease or condition.

The term “patient” means any mammal and preferably is a companion animal, such as a dog, cat or horse, or a human.

The terms “treating,” “treat,” “treatment,” as used herein, include curative, preventative (e.g., prophylactic) and palliative treatment.

The term “biomaterial” includes scaffolds, hydrogels, synthetic, artificial or natural materials which are biocompatible for use in a mammalian medical/surgical context.

The terms “controlled release,” “sustained release,” and similar terms used herein refer to the delivery of a compound or combination of compounds that ameliorates, attenuates or eliminates one or more symptoms of a particular disease or condition or prevents or delays the onset of one or more symptoms or a particular disease or condition over a predetermined period of time at a constant or variable rate, preferably a relatively constant rate, that maintains a concentration of active ingredient equivalent to a therapeutically effective amount over substantially all of the predetermined period of time.

The term “immobilized” used herein refers to any physical, chemical or biologically based means by which a molecule can be made immovable or fixed in place.

### Proteoglycans Useful in the Invention

Preferred embodiments of the invention use a proteoglycan comprising the core protein of domain I of a mammalian perlecan to which at least one glycosaminoglycan chain is attached. Preferred perlecan domain I proteins have the amino acid sequence of SEQ ID NO: 1 or 2.

Other proteins comprising perlecan domain I can also be used in the invention, such as the domain I contained in the sequence found at GenBank Acc. No. XM 513180 (GI: 55586414) (chimpanzee); the domain I from other perlecan sequences known in the art, and other domain I sequences identified from cDNA libraries using methods known in the art. To minimize immunological responses, it is preferred that the source of the perlecan be the same organism type as the intended recipient. The proteoglycan should have at least one and can have more glycosaminoglycan chains, varying in length or composition. More preferably, the proteoglycan is substituted with two or three glycosaminoglycan chains.

The proteoglycans useful in the invention include those molecules having conservative amino acid substitutions at one or more predicted non-essential amino acid residues when compared to a wild-type mammalian perlecan domain I. Substitutions may occur for example at sites not involved in GAG binding to the proteoglycan. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in the proteoglycan is preferably replaced with another amino acid residue from the same side chain family such that the proteoglycan retains the ability to bind growth factors.

In other preferred embodiments, the proteoglycan is a biologically active portion of the perlecan domain I that includes a domain or motif that has growth factor binding ability. Such domains or motifs include the domains associated with at least one glycosaminoglycan attachment to the core polypeptide. The invention also includes uses and compositions of proteoglycans in which the core protein comprises an amino acid sequence having at least about 70%, 80%, 90%, 95%, or 99% homology to the amino acid sequence of domain I of a mammalian perlecan, preferably to domain I of human perlecan and most preferably to SEQ ID NO:1, in which the core protein has attached at least one glycosaminoglycan chain.

Any of the well accepted methods of determining homology may be used. For example, homology may be calculated by use of the computer program GAP (UWGCG, University of Wisconsin, Genetic Computer Group, program algorithm of Needleman and Wunsch, J. Mol. Biol. 1970, 48, 443 453), setting the following parameters: TABLE-US-00001 Gap Weight: 12 Length Weight: 4 Average Match: 2.912 Average Mismatch: −2.003. Other computer programs that may be used are GAP, BESTFIT, BLAST, FASTA, and TFASTA in the Wisconsin Genetics Software Package, Version 8 (available from Genetics Computer Group (GCG), 575 Science Drive, Madison, Wis.).

The proteoglycan preferably has a size of less than 500 kDa, also preferably less than 200 kDa, also preferably less than 100 kDa, and also preferably less than 25 kDa. Larger molecules create difficulties with formulation and administration.

The proteoglycans used in the invention may be obtained in various ways, such as by chemical synthesis, isolation from perlecan, or recombinant production. Preferred is recombinant production. Examples of such production are found in Costell et al. (17). Costell et al. teaches preparation of perlecan domain I from mammalian cell clones on a preparative scale using the pRc/CMV expression vector sold by Invitrogen Corp. The expression vector was cotransfected together with plasmid pSV_{pac} into human embryonic kidney 293 cells and stable transfectants were selected with puromycin.

The proteoglycans of the invention may be used to induce differentiation to or maintenance of connective-tissue cells, particularly chondrocytes. The proteoglycans are used to bind and present heparin-binding growth factors.

### Growth Factors

For growth and proliferation of bone cells and cartilage cells the following growth factors may be useful in the devices and methods of the invention: (a) hedgehog proteins (b) transforming growth factors-beta (TGF-β super-family) including bone morphogenetic proteins (BMPs) which affect cell growth and proliferation, apoptosis and differentiation and induction of new gene expression, (c) bio-morphogenetic proteins which initiate the migration of mesenchymal cells and their differentiation to chondroblasts and chondrocytes and mineralization of cartilage, angiogenesis, osteoblast differentiation, bone formation and subsequently, remodeling of the bone, (d) fibroblast growth factors (FGF), (e) platelet derived growth factors (PDGF), (f) vascular endothelial growth factors, (g) epidermal growth factors, and the like. Preferred growth factors for attachment in vitro to the scaffolds and hydrogels of the invention are BMPs. Additional examples of suitable growth factors are included in U.S. Pat. No. 5,876,730 to Brigstock et al. issued Mar. 2, 1999 entitled “Heparin-binding growth factor (HBGF) polypeptides,” which discloses a group of heparin-binding growth factors isolated from uterine secretory fluids. Preferred are heparin-binding growth factors. Also preferred are growth factors from the fibroblast growth factor family, such as TGFB, FGF-2, BMP-2, and VEGF.

The growth factors can be present in the devices and biomaterials of the invention in a concentration of 1 nanogram per cubic centimeter to about 1 milligram per cubic centimeter. The choice of concentration may depend on the nature and form of the activity of the growth factor to be employed in each individual case, and on the nature of the scaffold material and its possibly inherent bioactivity. In one embodiment, the growth factor is BMP and is present in the device of the invention (such as the scaffold or hydrogel) within the range of 1 microgram per cubic centimeter to 100 micrograms per cubic centimeter.

### Sustained Release

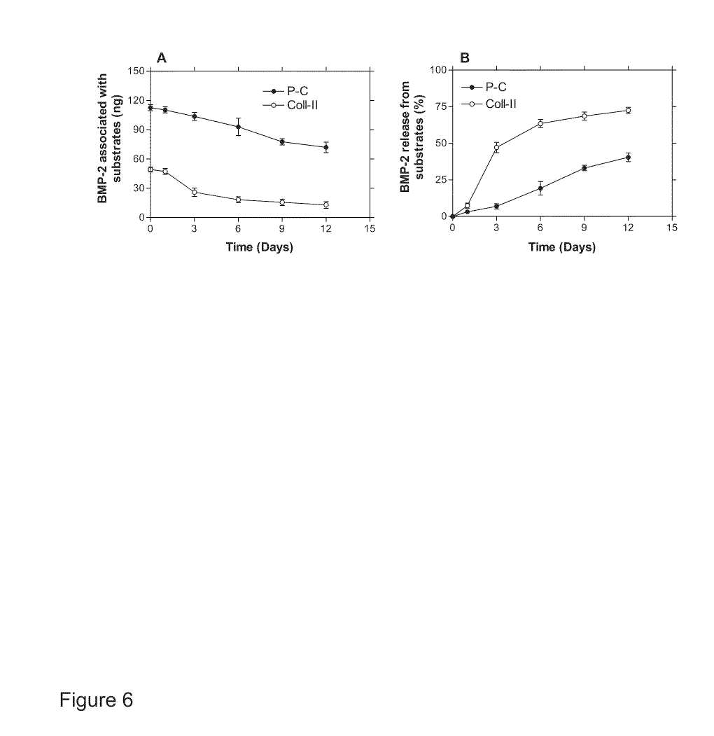

In one embodiment, sustained release is achieved by delivering an active agent such as a growth factor at therapeutically effective amounts over a fixed duration of time, such as, for example, over two, three, four, five, six or seven days or more with only one administration of the composition containing the active agent. Specifically, in one embodiment, the biomaterials and compositions of the invention provide for a release of bound growth factor of less than 25% of the growth factor over a predetermined period of three days, or less than 20%, or less than 15%, or less than 10% over three days. In other embodiments, the biomaterial releases 3 to 12% of the growth factor over three days, less than 60% of the growth factor over twelve days, less than 50% over twelve days, or 30 to 50% of the growth factor over twelve days. Such measurements of growth factor release may be made using any of the tests available to one skilled in the art, such as the in vitro test for release of growth factor reported in FIG. 6.

### Scaffolds

Devices coated with the proteoglycan-growth factor complex of the invention such as implants, scaffolds and/or hydrogels are also provided. The scaffold may be made of a polymer, a biologically derived material, ceramic, metal, or combinations thereof, that is biologically inert and physiologically compatible with mammalian tissues. Collagen is a preferred material for the scaffold. The scaffold/hydrogel material preferably does not induce an inflammatory response. The scaffold also preferably is capable of associating with the proteoglycan-growth factor complex at sufficient levels to satisfy the intended objective, e.g., ensure that a sustained release of an effective dose of growth factor is delivered over the desired time interval for proper tissue healing. The scaffold can immobilize the proteoglycan-growth factor complex covalently or non-covalently, such as by electrostatic charge or hydrophobic or hydrophilic interactions.

Preferred polymers are polyamides, polypeptides, polyesters, polycarbonates, polyurethanes, polyacetals, polysaccharides, and polyolefins. Specific examples of such polymers include silicone rubber, polyurethane rubber, polyethylene, polyvinyl chloride, poly(hydroxyethyl methacrylate), poly(methyl methacrylate), poly (ethyleneterephthalate), polypropylene, polystyrene, poly(tetrafluoroethylene), polyglycolic acid, cellulose, ethylcellulose, methycellulose, dextran, carboxymethylcellulose, hyaluronic acid, hydroxypropylmethylcellulose, nylon, collagen, and collagen-GAG. Additionally, the scaffold can be a copolymer, composite or blend of the above polymers.

The polymer may have other materials embedded in it, such as carbon fibers embedded in a polyurethane-poly(L-lactide matrix). Additional scaffold materials are known to those skilled in the art.

Preferred biologically derived materials are matrices comprised of collagen sponge, cortical bone chips, cancellous bone chips, cortico-cancellose bone chips, hydroxyapatite or like ceramics, bioactive glass, growth factors and demineralized bone, which are imbedded or suspended in a carrier material. The carrier material may be a fibrin-containing composition that coagulates, collagen formulations, hydroxylapatite, pleuronic polymers, synthetic or natural polymers, carboxymethylcellulose, gelatin, or combinations thereof. The carrier may be gelatin derived from human or animal tissue. Other useful biologically derived materials are mammalian tissues, such as perichondral tissue and periosteal tissue.

The proteoglycans may be used in soluble or insoluble form. The proteoglycan-growth factor complex may be a surface coating on a scaffold, such as surfaces used in tissue engineering or prosthetic devices. For example, scaffolds, hydrogels and medical devices may be coated ex vivo with the proteoglycan-growth factor complex and implanted in a mammalian body for sustained release of growth factor. In one embodiment, the proteoglycan-growth factor complex is further combined with collagen or attached to a collagen surface ex vivo and then implanted or injected into a mammalian body. The collagen itself may be the immobilizing biomaterial, or there may be an additional material useful for immobilization.

### Hydrogel

Hydrogels may be formed from a variety of polymeric materials and are useful in a variety of biomedical applications, such as direct injection of a therapeutic composition into bone joint. Hydrogels can be described physically as three-dimensional networks from hydrophilic polymers. Depending on the type of hydrogel, they contain varying percentages of water, but altogether do not dissolve in water. Despite their high water content, hydrogels are capable of additionally binding great volumes of liquid due to the presence of hydrophilic residues. Hydrogels swell extensively without changing their gelatinous structure. The basic physical features of hydrogel can be specifically modified, according to the properties of the polymers used and the additional special equipments of the products.

Preferably, the hydrogel is made of a polymer, a biologically derived material, a synthetically derived material or combinations thereof, that is biologically inert and physiologically compatible with mammalian tissues. The hydrogel material preferably does not induce an inflammatory response. The hydrogel material also preferably is capable of associating with the proteoglycan-growth factor complex at sufficient levels to satisfy the intended objective, e.g., insure a sustained release of an effect dose of growth factor is delivered over the desired time interval to for proper tissue healing. The hydrogel can immobilize the proteoglycan-growth factor complex covalently or non-covalently, such as by electrostatic charge or hydrophobic or hydrophilic interactions.

Examples of other materials which can be used to form a hydrogel include (a) modified alginates, (b) polysaccharides (e.g. gellan cum and carrageenans) which gel by exposure to monovalent cations, (c) polysaccharides (e.g., hyaluronic acid) that are very viscous liquids or are thiotropic and form a gel over time by the slow evolution of structure, and (d) polymeric hydrogel precursors (e.g., polyethylene oxide-polypropylene glycol block copolymers and proteins). U.S. Pat. No. 6,224,893 B1 provides a detailed description of the various polymers, and the chemical properties of such polymers, that are suitable for making hydrogels in accordance with the present invention.

The invention is also directed to hydrogels comprising the proteoglycan-growth factor complexes within the gel, as well as those coated with the complex as discussed above. For example, hydrogel monomers (natural or synthetic) are added to pharmaceutical compositions (with an initiator and, sometimes, cross-linking agents) and then allowed to polymerize, thereby encapsulating the complex within a hydrogel matrix. These techniques are used to provide microsphere carrier systems for drug targeting or controlled release systems. For example, cross-linked hydrogel microspheres have been used to encapsulate islet cells for the treatment of diabetes (Lim et al (1980) Science 210:908-910) or cancer cells that produce cancer-suppressing materials (U.S. Pat. No. 5,888,497), and peptides and proteins (Wang et al (1997) Pharm. Dev. and Technology 2:135-142).

### Collagen

Collagen serves as an immobilization substrate for the proteoglycan-growth factor complex to facilite a sustained release of an effective dose of growth factor delivered over the desired time interval for proper tissue healing. Collagen is the major protein comprising the ECM. The documented number of types of collagen vanes, but there are at least twelve. Types I, II and III are the most abundant and form fibrils of similar structure which are useful for the practice of the invention. However, depending upon the location of the wound, other types of collagen are envisioned as useful for the present invention. Collagen is a long, fibrous structural proteins that is tough and inextensible, with great tensile strength, but which can be easily and readily prepared for use in the invention as exemplified in the experimental section below and as understood by one of ordinary skill in the art. Any of the types of collagen may be used in the invention, such as collagen type I-XIII and any subtype of any of these types, such as type IIa. A particularly useful collagen for use in scaffolds and scaffold coatings is collagen type II and more particularly type IIa, and collagens that interact with collagen type II.

### Compositions

The invention also concerns compositions of proteoglycans and growth factors for injection into wound sites, such as hyaluron is administered today for cartilage therapy. Such compositions may be formulated with a pharmaceutically acceptable adjuvant as is known in the art. For example, aqueous formulations of the proteoglycan-growth factor complex may be made such that intraarticular injection is possible. One possibly composition comprises proteoglycan-growth factor complex in buffered physiological sodium chloride at a pH of 6.8-7.5. One sample formulation comprises the proteoglycan-growth factor complex; sodium chloride; monobasic sodium phosphate. 2H₂O; dibasic sodium phosphate.12H₂O and water for injection q.s. to 2.0 ml.

The compositions of the invention for injection may also comprise formulations of microspheres in which the microsphere contains the proteoglycan-growth factor complex and optionally collagen. The final composition may comprise proteoglycan-growth factor complex in an amount within the range of 0.1 to 100 mg/ml solution, 1.0 to 50 mg/ml solution, or 10-20 mg/ml solution. The composition also preferably contains a preservative, preferably selected from the group consisting of sodium benzoate, methylparaben, propyl paraben, and mixtures of sodium benzoate, methylparaben, and propyl paraben. The microspheres of the invention may be comprised of proteoglycan-growth factor complex in an amount of more than 20 weight % of the microsphere, and a biodegradable polymer selected from the group consisting of polylactic acid and poly(lactic-co-glycolic) acid, such as those polymers whose weight average molecular weight is in the range of 4,000 to 50,000. Other methods of making protein-containing microspheres are known and will be apparent to those in the art.

### Methods of Treatment

The present invention also concerns a method for treating a medical condition of the type that is characterized by the destruction of articular cartilage—preferably, joint injury, reactive arthritis, acute pyrophosphate arthritis (pseudogout), psoriatic arthritis, or juvenile rheumatoid arthritis, more preferably osteoarthritis, in a mammalian subject, preferably a human subject, which method comprises administering to the subject having the condition a therapeutically effective amount of the compositions of the invention or implanting a therapeutically effect amount of a device of the invention.

For the treatment of rheumatoid arthritis, the compositions and devices of the invention may be combined with other active agents, such as TNF-α inhibitors, such as anti-TNF monoclonal antibodies (such as Remicade®) and TNF receptor immunoglobulin molecules (such as Enbrel®), low dose methotrexate, lefunimide, hydroxychloroquine, d-penicilamine, auranofin or parenteral oral gold.

For the treatment of osteoarthritis, the administration of compositions and devices of the invention may be combined with treatment by administration of other recognized therapeutic agents, such as standard non-steroidal anti-inflammatory compounds, such as piroxicam, diclofenac, propionic acids, such as naproxen, flubiprofen, fenoprofen, ketoprofen, and ibuprofen; fenamates, such as mefenamic acid, indomethacin, sulindac, apazone, pyrazolones, such as phenylbutazone, salicylates, such as aspirin; COX-2 inhibitors, such as, celecoxib, valdecoxib, paracoxib and rofecoxib; analgesics, LTD-4, LTB-4 and 5-LO inhibitors, p38 kinase inhibitors and intraarticular therapies, such as corticosteroids and hyaluronic acids, such as hyalgan and synvisc.

The proteoglycan-growth factor complex of the invention can be administered directly to injured connective tissue, such as by implantation of a device or by direct injection, such as into the synovial fluid of the joint. The growth factors are attached to the proteoglycan ex vivo and then the immobilized proteoglycan-growth factor complex can be administered to damaged tissue, such as a bone fracture or cartilage tear. The growth factors will be released from the immobilized proteoglycan-growth factor complex in vivo at a therapeutic dosage level in sustained or controlled manner over time. Thereby tissue recovery will be enhanced.

The proteoglycan-growth factor complex is used to administer the growth factors as treatment for a variety of medical conditions over time. One important example is in the repair of bone, cartilage, or other cartilaginous connective tissue (such as tendon and ligament). Repair may be needed because of trauma, bone tumor resection, or in the case of joint fusion and spinal fusion for non-healing fractures and osteoporotic lesions. An immobilized proteoglycan-growth factor complex coated scaffold or hydrogel also may be used in treating tooth and jaw defects in cases of trauma, bone loss, tooth loss, and gum disease. The scaffolds also are useful in treating cartilage defects such as those which result from rheumatoid arthritis, osteoarthritis and trauma. The scaffolds also may be used to repair defects and damage in skin, muscle and other soft tissues such as results from trauma, burns, ulcers (diabetic ulcers, pressure sores, venus, stasis ulcers, etc.). Likewise, damage to visceral organs including liver damage, heart attack damage, and damage resulting from intestinal cancer or intestinal ulcer may be treated with the scaffolds of the invention.

Compositions of the proteoglycan-growth factor complex may also be injected directly into the site of cartilage damage, with or without the complex being immobilized on a biomaterial. The sustained release effect of the compositions of the invention is envisioned as allowing for an injection schedule that is not so frequent as to raise issues of patient compliance.

### EXAMPLES

The following discussion shows that domain I of perlecan functions as a sustained release carrier for growth factors necessary for chondrogenesis when immobilized. The experiments reported below show that PlnDI binds to both BMP-2 and collagen II fibrils via its GAG chains in a self assembly process. Thus, PlnDI offers a novel tool to enhance BMP-2 binding and function on scaffolds. As shown in the experiments, BMP-2 interacts with different substrates, including collagen II fibrils complexed with PlnDI. These interactions allow a sustained release of BMP-2 over time. Accordingly, PlnDI can improve substrate BMP-2 immobilization and release from scaffolds and/or fibrils, making it a prime candidate to mediate the sustained or controlled release of growth factors over time to effectively heal cartilaginous tissues or to prevent cartilaginous damage in joints at risk of developing osteoarthritis.

In addition the experiments show chondrogenic differentiation of C3H101/2 cells and primary mouse embryonic fibroblasts plated on these substrates or seeded in scaffolds modified with the substrates. Collectively, these findings show that PlnDI improves substrate BMP-2 immobilization onto scaffolds or fibrils and promotes chondrogenic differentiation.

### Materials and Methods

Heparinases I, II and III, chondroitinase ABC, testicular hyaluronidase, heparan sulfate (HS), chondroitin sulfate (CS), bovine serum albumin (BSA), Tween 20, D-(+)-glucose and collagen II from bovine tracheal cartilage (C1188) were obtained from Sigma-Aldrich, (St. Louis, Mo., USA). Recombinant human BMP-2 (rhBMP-2, 355-BM-010) and mouse monoclonal anti-human BMP-2 antibody (IgG2B, MAB3351) were obtained from R&D Systems, Inc. (Minneapolis, Minn., USA). Rat anti-heparan sulfate proteoglycan monoclonal antibody (directed against perlecan domain IV, MAB1948) and rabbit anti-aggrecan polyclonal antibody (AB1031) were purchased from Chemicon International Inc. (Temecula, Calif.). Rabbit anti-chicken tenascin polyclonal antibody was a generous gift from Drs. R. Chiquet-Ehrismann and T. Sakakura (Friedrich Miescher Institute, Switzerland). Rabbit anti-mouse collagen X polyclonal antibody (NC2 #90) was a generous gift from Dr. G. Lunstrum, (Shriners Hospital for Children). Rhodamine Red™-X-conjugated AffiniPure goat anti-rat IgG, sheep anti-mouse IgG conjugated HRP and normal rabbit serum were purchased from Jackson ImmuoResearch Laboratories, Inc. (West Grove, Pa.). Alexa Fluor®488 was obtained from Molecular Probes, Inc. (Eugene, Oreg.). Neutr-Avidin horseradish peroxidase conjugated (NeutrAvidin™-HRP), 3,3′,5,5′tetramethylbenzidine (TMB, 1-Step™ Ultra TMBELISA), blocking buffer (SuperBlock™ Blocking Buffer) and chemiluminescent substrate (SuperSignal West Dura Extended Duration Substrate) were purchased from Pierce Biotechology, Inc. (Rockford, Ill., USA). Polylactic acid (PLA) scaffolds were obtained from BD Biosicences (BD™ Three Dimensional OPLA® scaffolds). rhBMP-2 to PlnDI Binding.

A dot blotting format was employed to determine if PlnDI was functionally active in binding rhBMP-2 (7). Recombinant mouse PlnDI (12 μg) was digested with heparinases I, II, and III in PBS containing 1 mM Ca²⁺ and Mg²⁺ for 4 h at 37° C. Digested and undigested PlnDI (3 μg) were blotted onto nitrocellulose, and subsequently blocked with 5% (w/v) fat-free milk powder in blocking buffer (SuperBlock™, Pierce Biotechnology, Inc.) for 1 h at room temperature. After washing with blocking buffer, 100 ng of rhBMP-2 was added to each well of the blotting apparatus and incubated for 4 h at room temperature. The membrane then was removed from the blotting apparatus, and blocked with 3% (w/v) BSA in blocking buffer for 1 h at 4° C., prior to incubation in 2.0 μg/ml of monoclonal mouse antihuman BMP-2 antibody in block buffer overnight at 4° C. After washing five times at room temperature with 0.05% (v/v) Tween 20 in PBS (PBS-T), the membrane was incubated with sheep anti-mouse IgG conjugated HRP (1:200,000) in blocking buffer for 1 h at room temperature. Following this incubation the membrane was washed again in PBS-T. The bound antibody was detected via enhanced chemiluminescence. The binding of rhBMP-2 to PlnDI was evaluated by densitometry and expressed as individual density values (IDV).

Preparation of Microplate Coating with Collagen II Fibrils

Freeze-dried collagen II extracted from bovine tracheal cartilage was dissolved at 4 mg/ml in 0.5 M acetic acid for 48 h at 4° C. to make collagen II dispersions (collagen II monomers). Collagen II fibrils were formed by dialyzing 2.5 ml of collagen II acid dispersion against 1 L of PBS (pH 7.4) for 48 h at room temperature, and then incubating for 24 h at 37° C. as described previously (23). In vitro fibril formation was monitored by the increase in absorbance at 400 nm (24, 25). The collagen II fibril preparation then was diluted with PBS to 1.0 mg/ml and stored at 4° C. Denatured collagen II fibrils were obtained by heating collagen II fibril preparations at 60° C. for 30 min as previously described (23). To immobilize collagen II into plastic plates, each well of 96-well micro-plates was incubated with 10 μg of collagen II fibrils, or denatured collagen II fibril suspension or acid dispersion (collagen II monomer) in 100 μl for 24 h at 37° C. Control wells were coated with 100 μl of 100 μg/ml BSA solution in PBS. After rinsing with PBS, the coated 96-well plates were stored at 4° C. for future use. Collagen II coating efficiency was determined by measuring hydroxyproline content of the coated well surfaces (48). All collagen forms used gave similar coating efficiencies (+/−5%).

### PlnDI Binding to Collagen II Fibrils

To determine if PlnDI bound to collagen II fibrils, a solid-phase binding assay was performed essentially as described previously (23-25). Briefly, PlnDI was biotinylated with Sulfo-NHS-LC-Biotin using EZ-Link™ Sulfo-NHS-LC-Biotinylation Kit (Pierce Biotechology, Inc, Rockford, Ill., USA), according to the manufacturer's instructions. The association of biotinylated PlnDI with collagen II fibrils immobilized in microplates was determined by binding of NeutrAvidin conjugated horseradish peroxidase (NA-HRP).

After blocking with 3% (w/v) BSA in PBS, 100 μl of biotinylated PlnDI in blocking buffer was added at increasing concentrations (0-600 μg/ml) to each well of a 96-well microplate and incubated for 2 hr at room temperature. After washing three times with PBS, the bound biotinylated PlnDI was incubated with NA-HRP (0.1 μg/ml) in 100 μl blocking buffer for 30 min at room temperature. The wells finally were incubated with 200 μl of TMB solution followed by washing with PBS. The reaction was stopped with 500 μl of 2M sulfuric acid. The optical density was measured at 450 nm. The same assay was used to assess interactions of biotinylated PlnDI with denatured collagen fibrils and collagen II monomers.

The specific binding of biotinylated PlnDI to collagen II fibrils was evaluated further by competitive binding of unlabeled PlnDI. In the assay, 3 μg of biotinylated PlnDI was added to collagen II fibril-coated wells in the presence of increasing molar ratios of unlabelled PlnDI/biotinylated PlnDI (from 0 to 40). The association of biotinylated PlnDI with collagen II fibrils was measured as described above.

To investigate to what extent the protein and GAG constituents of PlnDI mediated interactions with collagen II fibrils, biotinylated PlnDI (3 μg), digested or undigested with heparinases I, II and III and chondroitinase ABC, was added into each well of collagen II fibril-coated microplates. HS (25 μg/well) or CS (25 μg/well) were used to compete for biotinylated PlnDI binding (6 μg/well) to collagen II fibrils. Binding characteristics of biotinylated PlnDI, following digestion of HS or CS or in competition with HS or CS, were evaluated as described above.

Binding of rhBMP-2 to P-C Fibrils

After immobilizing collagen II fibrils into 96-well microplates and blocking with 3% (w/v) BSA in PBS, PlnDI (3 μg/well), undigested or digested with heparinases I, II and III or chondroitinase ABC, was incubated with the collagen II fibrils resulting in the following substrates: PlnDI-collagen II fibrils (P-C fibrils), heparinases I, II and III digested P-C fibrils and chondroitinase ABC-digested P-C fibrils. Surfaces coated with collagen II fibrils alone or BSA (BSA) served as controls. Solid-phase binding assays were employed to assess rhBMP-2 binding. In this experiment, rhBMP-2 (50 ng) in blocking buffer was added to each well and incubated for 2 h at room temperature. After washing three times with PBS, anti-human BMP-2 antibody conjugated to HRP and colorimetric reagents of the BMP-2 Quantikine ELISA Kit (R&D System, Mc. Minneapolis, Minn.) were used to identify the rhBMP-2 associated with these substrates, according to manufacturer's instructions.

Quantification of rhBMP-2 Release

The release kinetics of rhBMP-2 from P-C fibrils or collagen II fibrils, were measured using a sandwich ELISA. In 4-well plates (Nalge-Nunc International; Rochester, N.Y.), collagen II fibrils (100 μl in 300 μl of PBS) were added to each well and then incubated with either PBS or PlnDI (9 μg in 300 μl of PBS/well) to form substrates of collagen II fibrils alone or P-C fibrils, as described above. The coated 4-well plates were sterilized under UV irradiation in a standard tissue culture hood for 2 h. After washing with PBS, 200 ng of rhBMP-2 in 300 μl of release buffer (DMEM containing 1% (w/v) BSA, 100 U/ml penicillin and 100 μg/ml streptomycin) was added into each well, and incubated with the substrates for 2 h at 37° C. rhBMP-2 in the release buffer was determined in 0.8 ml collected at day 0. Next, release buffer (0.8 ml) was added into each well after which it was retrieved at 1, 3, 6, 12 days, and stored at −40° C. The content of rhBMP-2 in the release buffer was determined with a sandwich ELISA assay kit (Quantikine BMP-2 ELISA, R&D Systems, Inc, Minneapolis, Minn.), according to the manufacturer's instructions. The content of rhBMP-2 associated with each substrate, and the percent of rhBMP-2 released from the substrates were calculated.

### High Density Micromass Cultures

The multipotential mouse embryonic fibroblast stem cell line, C3H10T1/2, was obtained from the American Type Culture Collection (ATCC, Rockville, Md.) and cultured in DMEM/F12 containing 10% (v/v) FBS, 100 U/ml penicillin and 100 μg/ml streptomycin, at 37° C. in a humidified atmosphere of air: CO₂, 95:5 (v/v). High density micromass culture of C3H10T1/2 was employed as described previously (46, 49). P-C fibrils and collagen II fibrils only substrates were pre-coated on 4-well plates as described above, and then incubated with rhBMP-2 (200 ng/well) in 300 μl of DMEM containing 5% (v/v) FBS for two h at room temperature to form P-C-B fibrils and C-B fibrils. After washing with PBS two times and sterilizing with UV irradiation for 30 min, the 4-well plates loaded with different substrates were air-dried in a laminar-flow hood, and then C3H10T1/2 cells were spot-seeded as 10 μl drops containing 1×10⁵ cells, in the center of each well. After cells had attached for 1-2 h at 37° C., 0.8 ml of chondrogenic differentiation medium (CMRL-1066 containing 15% (v/v) FBS, ascorbic acid (50 μg/ml), citrate (50 μg/ml), pyruvate (50 μg), 100 U/ml penicillin and 100 μg/ml streptomycin [14, 19, 20]), was added to each well. The medium was changed every 2 days. Cultures were maintained at 37° C. in a humidified atmosphere of air: CO₂, 95:5 (v/v) until harvest.

### Alcian Blue Staining for Micromass Culture

To observe chondrogenic differentiation of C3H10T1/2 cells on different substrates, Alcian blue staining was performed as described previously (21, 46, 49, 50). Briefly, after 6 days of micromass culture, cells were rinsed with PBS, fixed with 10% (v/v) formalin containing 0.5% (w/v) cetylpyridinium chloride (CPC) for 10 min at room temperature, briefly rinsed with 3% (v/v) glacial acetic acid (pH 1.0) and then incubated in 1 ml of 0.5% (w/v) Alcian blue 8GX (Sigma) in 3% (v/v) glacial acetic acid (pH 1.0) overnight at room temperature.

Collagen II, Aggrecan and SOX 9 mRNA Expression

RNA was extracted from C3H10T1/2 cell micromass cultures at day 6. Each sample was comprised of four micromass cultures collected in cell lysis buffer from the RNeasy Mini Kit (QIAGEN; Valencia, Calif.), and passed through a Qiashredder homogenizer (QIAGEN) and Qiashredder spin column according to the manufacturer's protocol. Isolated RNA was treated using the DNA-free Kit (Ambion, Austin, Tex.) and quantified spectrophotometrically. cDNA was generated from RNA using random hexamers and RNase inhibitor from GeneAmp RNA PCR Core kit (Applied Biosystems, Forster City, Calif.), and reverse transcriptase, dNTPs and RT buffer from the Omniscript RT Kit (QIAGEN) according to the manufacturer's protocol. rnRNA levels were determined using real-time quantitative PCR, performed using SYBR Green PCR Master Mix (Applied Biosystems, Warrington WA1 4SR, UK). PCR reactions were performed and monitored using ABI Prism 7700 Sequence Detection System (AB Applied Biosystems, Foster City, Calif.) with a two step cycling protocol (annealing and elongation at 60° C., and denaturation at 94° C.). The levels of expression of mRNA were calculated with the comparative threshold cycle (Ct) method with 2^{-ΔΔCt} formula (User Bulletin No. 2, BI Prism 7700 Sequence Detection System). The Ct value of each target sequence was subtracted from the Ct value of β-actin, to derive ΔCt. The calculation of ΔΔCt involved subtraction of the ΔCt value of C3H10T1/2 cells cultured on uncoated plates. The validation experiment demonstrated that the amplifying efficiency of the targets (collagen II, aggrecan and sox9) and reference (β-actin) were approximately equal (slope difference <0.1). Each sample was assessed in triplicate. Specificity of primers was verified by dissociation of amplicons. The primer pairs used for PCR reactions are listed in table 1.

### Preparation of ECM Modified-PLA Scaffolds and PlnDI/Collagen II Fibril-PLA Scaffolds

Collagen II fibril-PLA scaffolds were prepared by coating collagen II fibrils on PLA sponges as described previously (35, 51) with some modification. The PLA sponges (average pore size: 100-200 μm, hydration capacity: 30 μl, diameter: 4.2-5.2 mm, height: 3.9-4.5 mm, volume: 0.039 cm³) were immersed in collagen II fibril solution (1.0 mg/ml in PBS) containing D-(+)-glucose (9 mM) and submitted to constant rotary agitation overnight at 4° C. The collagen II fibril-containing PLA sponges then were frozen at −80° C. for 24 h, and subsequently lyophilized for an additional 24 h. The lyophilized collagen II fibril-PLA scaffolds were UV cross-linked as described previously using a UV crosslink chamber (Stratalinker 2400™, Stratagene Cloning Systems, La Jolla, Calif., USA). To further fabricate P-C fibril-PLA scaffolds, collagen II fibril-PLA scaffolds were incubated with PlnDI (30 μg/ml) with constant rotary agitation for 2 h at room temperature. The structure of the scaffolds was observed employing scanning electron microscope (SEM).

rhBMP-2 Binding to Scaffolds

To investigate the binding of rhBMP-2 to various scaffolds, an ELISA was employed. After blocking with 3% (w/v) BSA in PBS, PLA, collagen II-PLA or P-CPLA scaffolds were incubated with rhBMP-2 (200 ng/ml) with constant rotary agitation for 2 h at room temperature, and then washed 3 times with PBS-T on shaker at room temperature to remove unbound rhBMP-2. rhBMP-2 binding to scaffolds was measured with the BMP-2 Quantikine ELISA Kit (R&D System, Inc. Minneapolis, Minn.) according to the manufacturer's instructions. Each scaffold was further reacted with 3 ml of ELISA kit color reagent and then dried with a Kaydry wiper (Kimberly-Clark, Co., Roswell, Ga., USA) to stop the reaction and immediately photographed. In addition, after addition of stop buffer 200 μl of the reactant solution was transferred to wells of 96-well plates for absorbance measurement at 450 nm.

Seeding Scaffolds with Cells and 3D Tissue Culture

Scaffolds, polylactic acid (PLA), collagen II-PLA and P-C PLA] were incubated with rhBMP-2 (200 ng/ml in PBS) for 2 h. These coated scaffolds were immersed in 20 ml of CMRL-1066 medium containing 10% (v/v) FBS, and then briefly dabbed with a sterile gauze to remove excess medium. To form cell-scaffold constructs, dynamic seeding was used to load C3H10T1/2 cells onto scaffolds, according to the manufacturer's instruction (BD™ Three Dimensional OPLA® Scaffold, Guidelines for Use, BD Biosciences) as reported previously (52). Cell-scaffold constructs on PLA, collagen II-PLA and P-C PLA served as controls. Three scaffolds of each type were placed into 50 ml conical tubes (BD Falcon Conical Centrifuge Tubes) and then incubated in 1 ml of C3H10T1/2 cells suspension (2×10⁷ cells/ml) in CMRL-1066 medium containing 15% (v/v) FBS. The tubes were placed on an orbital shaker (Lab-Line Instruments. Inc. Melrose Park, Ill.) and rotary agitated in an incubator at 37° C. in a humidified atmosphere consisting of air: CO₂, 95/5 (v/v) at 250 rpm for 3 h. To maintain appropriate pH during extended incubation times, 4 ml of fresh CMRL-1066 medium containing 10% (v/v) FBS was added to each tube, and then the tubes were agitated for additional 12 h under the same conditions. After gently washing with CMRL-1066 media to remove non-adherent cells, the cell-seeded scaffolds were transferred into 25 cm² cell culture flasks (Corning Incorporated, Corning, N.Y.) and incubated in 8 ml of CMRL-1066 media, containing 15% (v/v) FBS, ascorbic acid (50 μg/ml), citrate (50 pyruvate (50 μg), 100 U/ml penicillin and 100 μg/ml streptomycin, at 37° C. in a humidified atmosphere of air: CO₂, 95:5 (v/v). Finally, dynamic culture was performed by placing the cell culture flask, fixed in a specially designed stand with an up-standing position, on the orbital shaker at 200 rpm in the incubator. The media was changed every 3 days. After 21 days of culture, cell-scaffold constructs were harvested for morphological analysis. Mouse embryonic fibroblasts (MEFs) were isolated from day 14 post coitum embryos of ICR mice using established methods (43). Differentiation experiments were carried out using cells between passage 3 and 4. Constructs of MEFs-scaffold were formed and cultured with same method used for C3H10T1/2 cells-scaffold constructs. As noted above, in each experiment three separate scaffolds were prepared in each test group and each experiment performed three times with similar results.

### Histological and Immunohistochemical Analysis

The cell-scaffold constructs were rinsed with PBS, fixed for 2 hr in 10% (w/v) formalin, dehydrated through a graded series of ethanol and, embedded in paraffin. Thick sections (10 μm) were cut through the center of scaffolds for Safranin O/Fast Green and von Kossa staining. For cryosectioning, the cell-scaffold constructs were embedded in O.C.T. (Sakura Finetek, Torrance, Calif.) frozen on dry ice. Sections of 30 μm thickness were cut through the center of cell-scaffold constructs for alkaline phosphatase (ALP), Oil Red staining and immunohistochemical analysis. The staining procedures of Safranin O/Fast Green, von Kossa, ALP and Oil-Red were performed according to standard histological protocols (33, 43, 53-55). For immunohistochemical analysis, crysections were fixed with 4% (w/v) paraformaldehyde in PBS for 30 min at room temperature followed by digestion with chondroitinase ABC (2.5 U/ml) for aggrecan staining, or with 0.25% (w/v) testicular hyaluronidase for Pln, tenascin and collagen X staining, for 1 h at 37° C. The specimens were blocked with DAKO® serum-free protein block (DAKO Co., Carpinteria, Calif.), and incubated with primary antibodies against aggrecan (rabbit antiaggrecan polyclonal antibody, 1:50), perlecan (rat anti-perlecan domain IV monoclonal antibody, 1:60), tenascin (rabbit anti-tenascin polyclonal antibody, 1:100) or collagen X (rabbit anti-collagen X, 1:200), respectively, for 1 h at 37° C. After rinsing with PBS, sections then were incubated with secondary antibodies of Alexa Fluor®488 goat antirabbit (1:500) for aggrecan detection, Rhodamine Red™-X-conjugated affiniPure goat anti-rat IgG (1:100) for perlecan detection or Alexa Fluor 568 goat anti-rabbit (1:50) for tenascin and collagen X detection for 1 h at 37° C. Sections then were rinsed three times with PBS, placed under glass coverslips and observed and photographed using confocal microscopy.

### Statistical Analysis

Unless otherwise stated, all values are expressed as means±standard deviations (SD) and one-way ANOVA. All studies were assayed using samples from separate determinations in triplicate. Statistical significance was determined by a Tukey-Kramer multiple comparisons test; p values <0.01 were considered significant.

### Results:

rhBMP-2 Binding to PlnDI

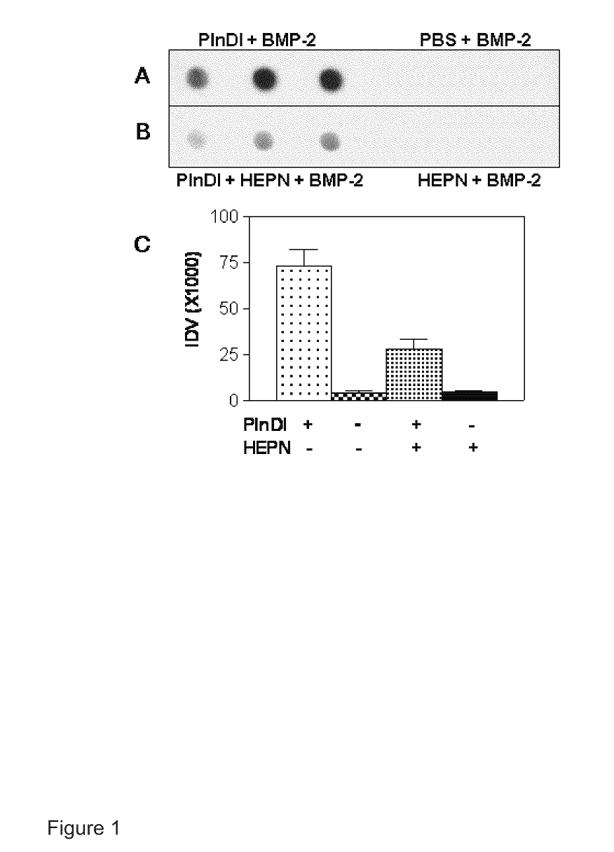

A photograph of a representative dot blot depicting rhBMP-2 binding to PlnDI is shown in FIG. 1A, B and demonstrates the heparan sulfate dependence, i.e., heparinase sensitivity, of the interaction. The densitometric quantitation of these data is summarized in FIG. 1C. Together, these data demonstrate that PlnDI binds rhBMP-2 robustly compared to negative controls (BMP-2+PBS and BMP-2+ heparinase) (P<0.001). In addition, HS chains attached in PlnDI are largely responsible for rhBMP-2 since heparinase treatment greatly reduced the binding of rhBMP-2 (P<0.001).

### Binding of PlnDI to Collagen II Fibrils

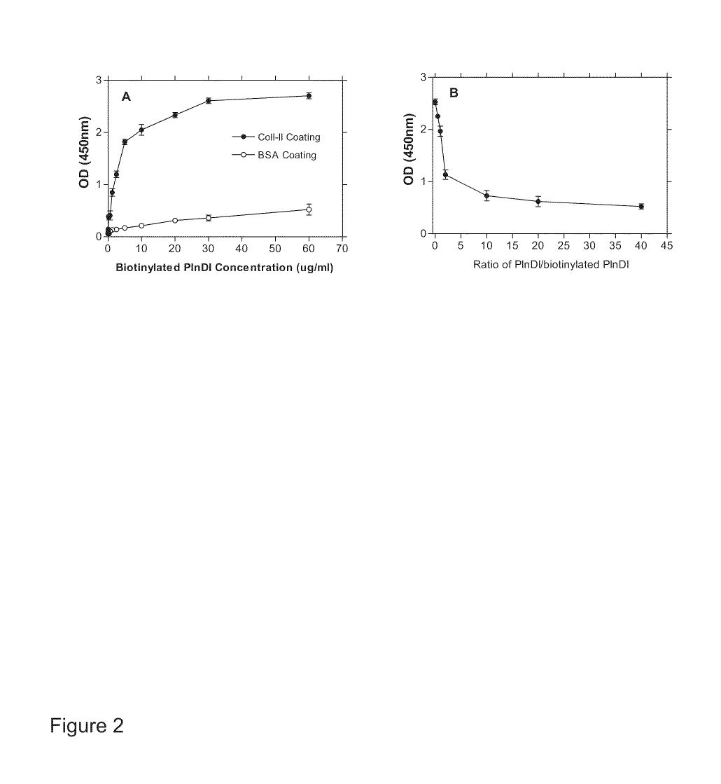

The solid-phase assays provided a simple, quantitative assay for detection of protein binding to collagen II. Initially, immobilized collagen II fibrils were incubated with soluble, biotinylated PlnDI to determine if PlnDI could bind to collagen II fibrils. Biotinylated PlnDI interacted with collagen II fibrils in a saturable manner, as expected for specific binding. In contrast, biotinylated PlnDI bound poorly to BSA-coated surfaces and represented a nonspecific binding control (P<0.001, FIG. 2). In addition, biotinylated PlnDI binding to collagen II fibrils saturated at concentrations of approximately 10-20 μg protein/ml, i.e., approximately 45-900 nM with half-saturation occurring at approximately 2.5 μg protein/ml, i.e., approximately 110 nM (FIG. 2A). As an additional specificity control, unlabeled PlnDI was used to compete for the biotinylated PlnDI binding. Biotinylated PlnDI binding to collagen II fibrils was blocked >80% in a dose dependent fashion by unlabeled PlnDI (FIG. 2B), suggesting that most binding was due to interactions with PlnDI and not biotin.

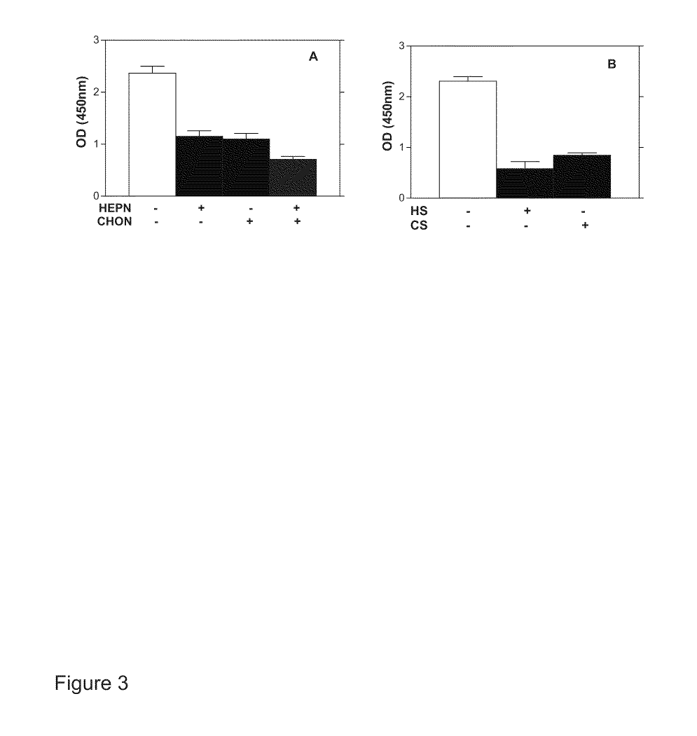

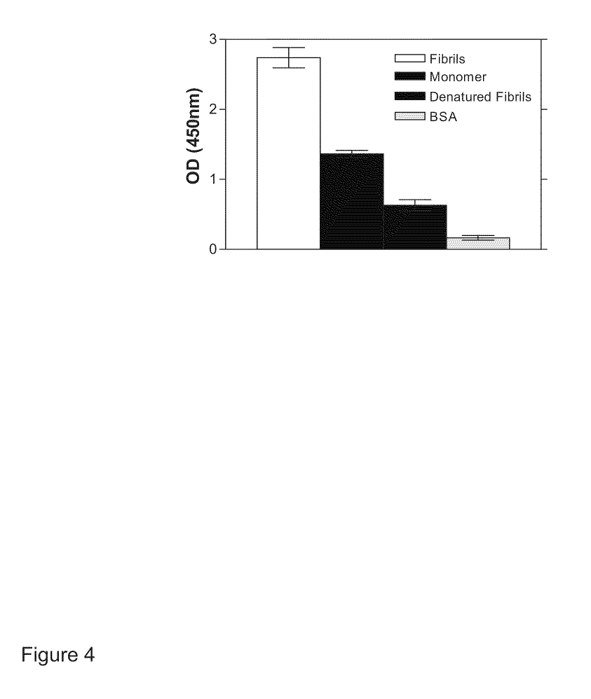

Next, collagen II fibril-coated plates were incubated with biotinylated PlnDI that had been predigested with heparinases I, II and III or chondroitinase ABC. Binding was reduced significantly by predigestion with either heparinase or chondroitinase with maximal inhibition observed with combined predigestion (P<0.001; FIG. 3A). Similar results were obtained in another type of experiment in which excess soluble HS or CS was used to compete for binding sites in collagen II fibrils (P<0.001; FIG. 3B). These results demonstrated that both HS and CS of PlnDI contribute to binding to collagen II fibrils. To determine if the physical form of collagen II impacted PlnDI binding, 96-well microplates were coated with natural collagen II fibrils, heat-denatured fibrils and collagen II monomers. The results demonstrated that significantly more biotinylated PlnDI bound to native fibrils than either denatured fibrils or monomers (P<0.001; FIG. 4). Collagen II monomers bound significantly more PlnDI than denatured fibrils (P<0.01). rhBMP-2 binding to P-C fibrils

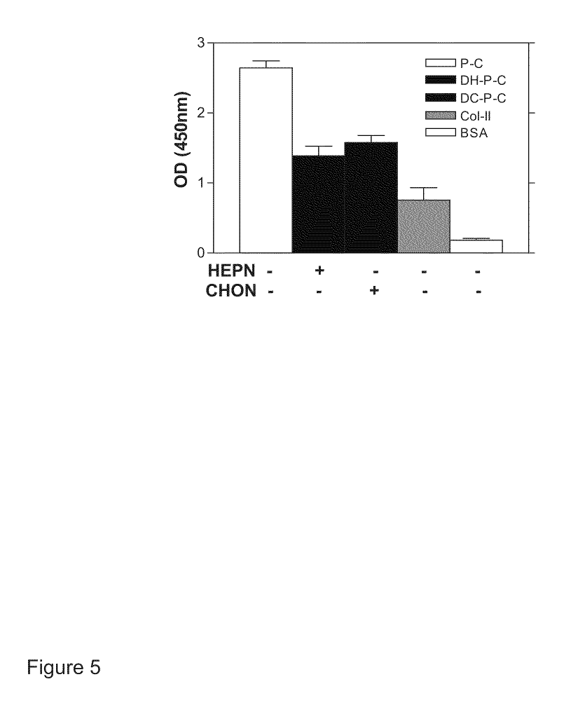

For determining if rhBMP-2 could bind to P-C fibrils, a solid phase binding assay was used. P-C fibril complexes bound significantly more rhBMP-2 than collagen II fibrils alone (P<0.001; FIG. 5). Digestion of PlnDI with either heparinase (DHP-C) or chondroitinase (DC-P-C) significantly reduced rhBMP-2 binding to P-C fibril complexes, although the residual binding was still significantly greater than to collagen fibrils alone (P<0.001). Thus, as was the case for PlnDI binding to collagen II fibrils, both HS and CS GAG chains contributed greatly to binding rhBMP-2 to P-C fibril complexes. rhBMP-2 release kinetics

rhBMP-2 release from P-C fibril complexes and collagen II fibrils alone was evaluated in vitro by incubation of these substrates in a physiological buffer for up to 12 days (FIG. 6). rhBMP-2 release was quantified using a sandwich ELISA. P-C fibril complexes initially bound 112 ng±4 of rhBMP-2 (day 0) in contrast with collagen II fibrils alone that bound only 49 ng±3 of rhBMP-2 (day 0). After 3 days, P-C fibrils retained 103 ng±4 of rhBMP-2 (FIG. 6A), releasing only 7.3%±3.4% of initially bound rhBMP-2. In contrast, at the same time collagen II fibrils alone retained 26 ng±6 of rhBMP-2 releasing 47.7%±4.9% of initially bound rhBMP-2 (FIG. 6B). After 12 days of incubation, P-C fibril complexes retained 72 ng±5 rhBMP-2 (FIG. 6A) releasing 41.5%±5.7% of initially bound rhBMP-2. At this time point, collagen II fibrils alone retained very little, i.e., 13 ng+3, rhBMP-2 releasing 71.3%±3.7% of initially bound rhBMP-2 (FIG. 6B). These findings demonstrated that P-C fibril complexes not only immobilized significantly more rhBMP-2, but also retained the HBGF well during extended incubation in physiological buffer.

### C3HT101/2 Chondrogenic Differentiation

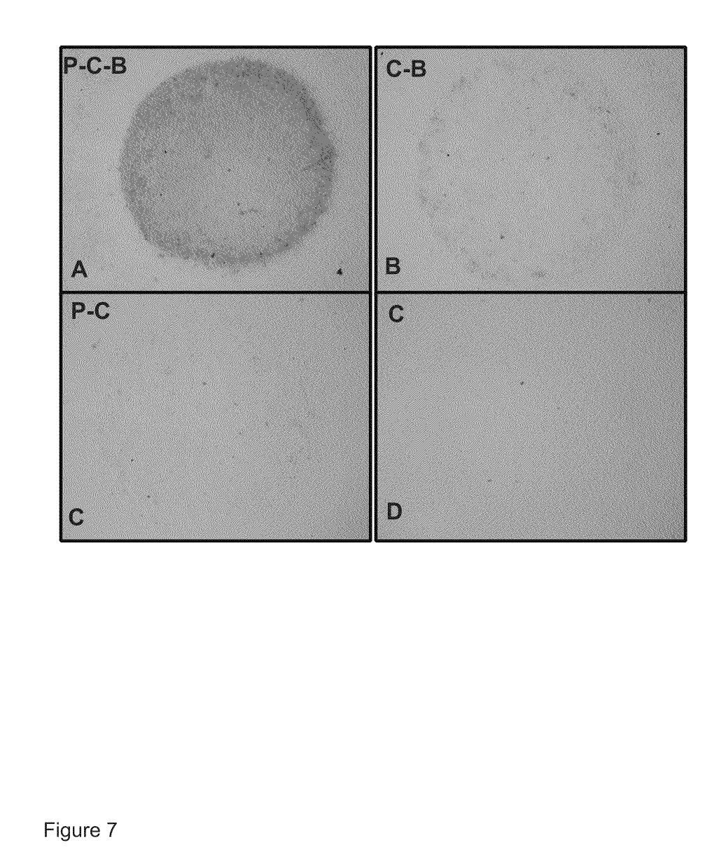

C3H10T1/2 cells were placed in micromass cultures on collagen II fibrils (C), P-C fibril complexes, collagen II fibrils with bound rhBMP-2 (C-B fibrils) or P-C-B fibrils. After 6 days of culture, they were stained with Alcian blue as an index of chondrogenic differentiation, i.e., GAG accumulation (FIG. 7). Micromass cultures displayed positive staining when plated on both P-C-B and C-B fibrils, and negative staining when plated without BMP-2; however, Alcian blue staining of micromass cultures on P-C-B fibrils was much more robust than on C-B fibrils in the absence of PlnDI.

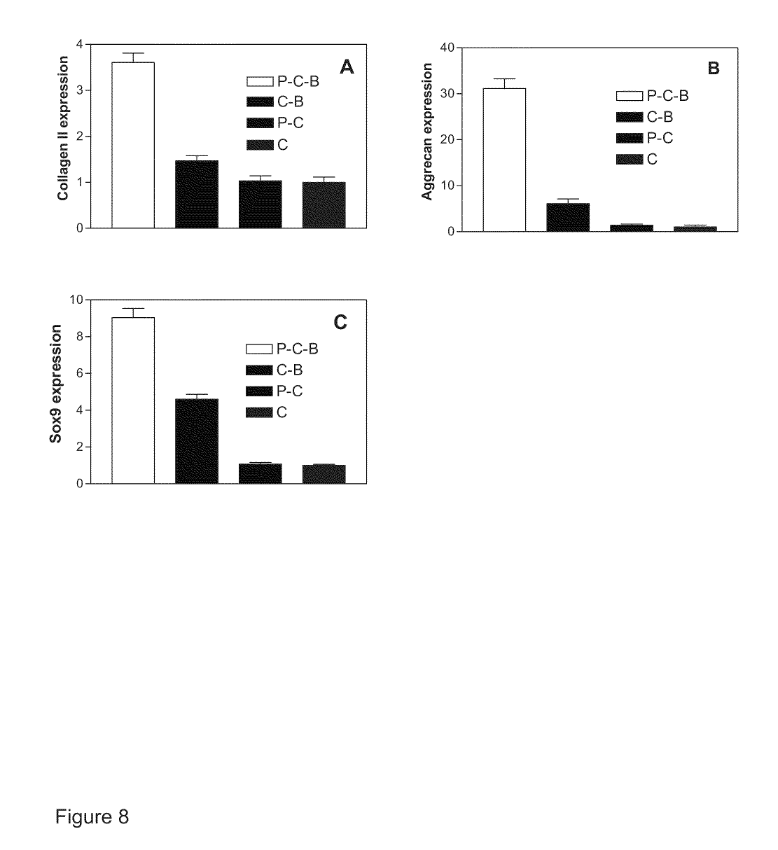

Chondrogenic differentiation also was evaluated by examining chondrocyte-marker gene expression by real time PCR (FIG. 8). Collagen II, aggrecan and sox9 mRNA content were normalized to β-actin mRNA in each sample. Expression of all three marker mRNAs was highest when micromass cultures were plated on P-C-B fibrils (P<0.001); however, marker mRNA expression was higher for micromass cultures plated on C-B fibrils than on either other matrix in the absence of BMP-2 (P<0.01). No significant difference in marker mRNA expression was found between collagen II fibrils with or without PlnDI without BMP-2 (P>0.05). These results demonstrate that all components of P-C-B fibril complex are required to support optimal C3H10T1/2 chondrogenic differentiation in high-density micromass culture, and that addition of BMP-2 is necessary regardless of matrix.

### Physical Properties and Binding Capacity of Different Scaffolds

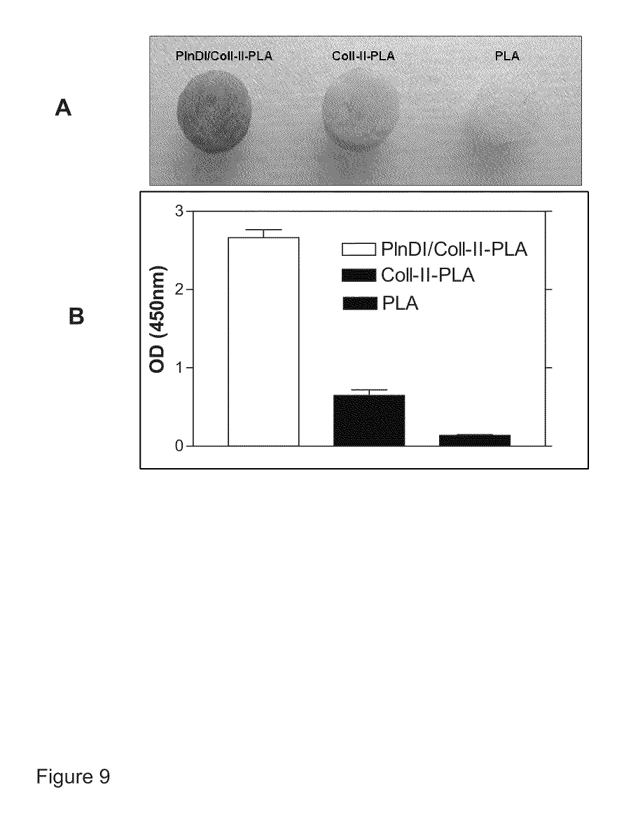

Scanning electron microscopy revealed that scaffolds of collagen II fibrils/PLA and P-C fibrils-PLA maintained the porous structures normally observed with uncoated PLA scaffolds [49; data not shown]. An ELISA-based assay was used to index rhBMP-2 binding to the various scaffolds. FIG. 9 shows that P-C fibril-PLA scaffolds displayed the highest rhBMP-2 binding; however, collagen II fibril/PLA scaffolds also displayed binding significantly above that of PLA alone, albeit much lower than that of P-C fibril-PLA scaffolds.

### Histochemical and Immunohistochemical Analysis

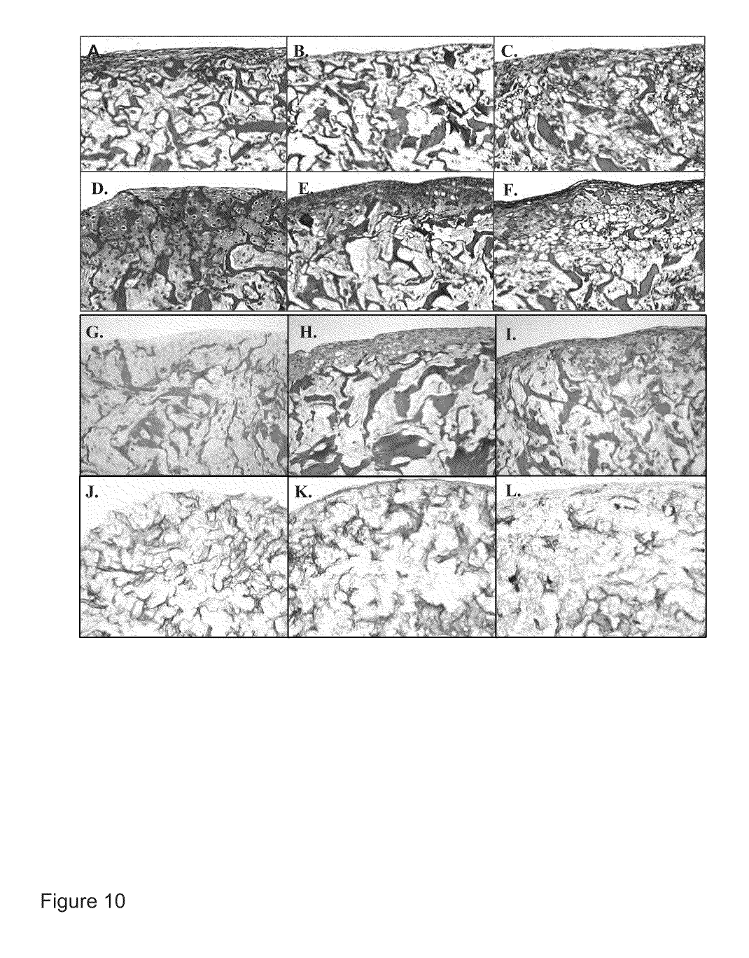

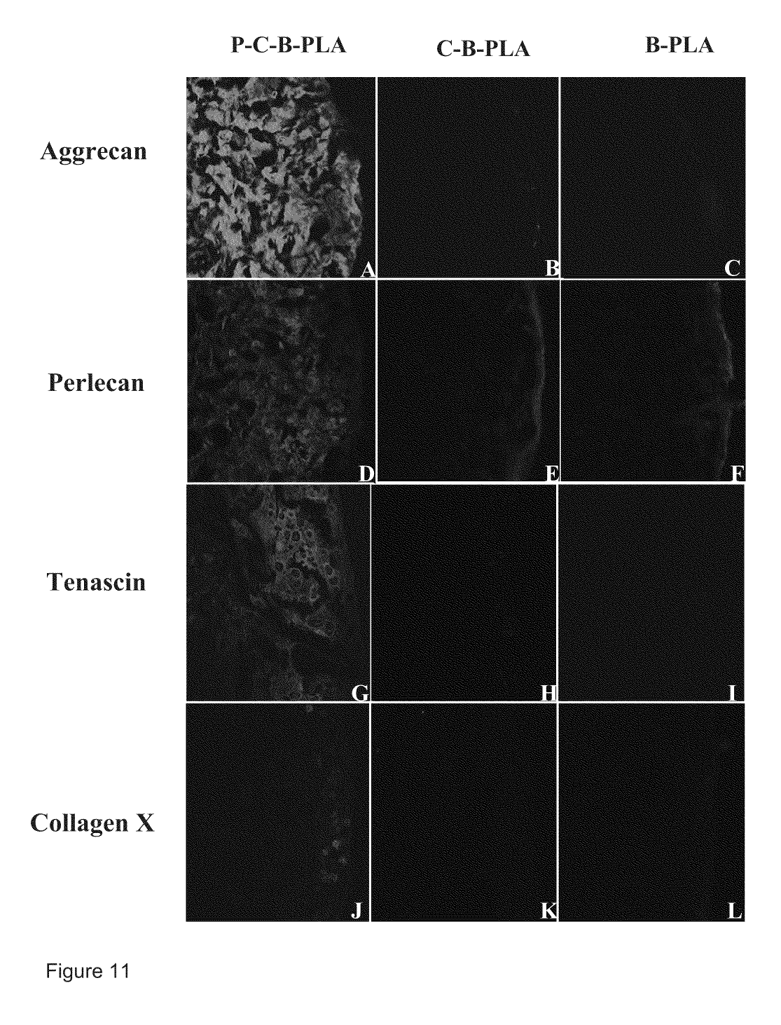

The morphology of cell-scaffold constructs was examined histologically with Safranin O/Fast Green staining, which stains negatively charged GAGs red and nuclei dark purple/black. Sections from P-C-B-PLA scaffolds were strongly positive for GAG compared with other constructs, and revealed round chondrocyte-like cells embedded in lacunae and surrounded by abundant ECM (FIG. 10D). Sections from C-B-PLA scaffolds revealed fibroblast-like cells embedded in compacted ECM that thickly covered the exterior scaffold surface (FIG. 10E). Cells seeded into BMP-2-PLA (FIG. 9F), P-C-PLA (FIG. 9A), collagen II-PLA (FIG. 10B) and PLA alone (FIG. 10C) scaffolds demonstrated no obvious cartilage-like tissue. Alkaline phosphatase staining for chondrocyte maturation (FIG. 10G-I) showed weak staining in some regions of P-C-B-PLA scaffolds (FIG. 10G), but none in the other constructs. Von Kossa staining of cell-scaffold constructs showed that no mineralized ECM was present in any of the scaffold constructs (FIG. 10J-L). Immunohistochemical staining for the cartilage ECM markers, aggrecan, Pln and tenascin, showed strong positive staining in P-C-B-PLA scaffolds (FIG. 11A, D and G) with weak to no signal in the other scaffolds. A thin layer of Pln was evident at the exterior surface of collagen II-BMP2-PLA and BMP-2-PLA scaffolds (FIGS. 11E and F). Staining for collagen X, a marker of late hypertrophic chondrocyte differentiation only was found in isolated regions of P-C-B-PLA scaffolds (FIG. 11J), but was virtually absent in the other scaffolds (FIGS. 11K and L).

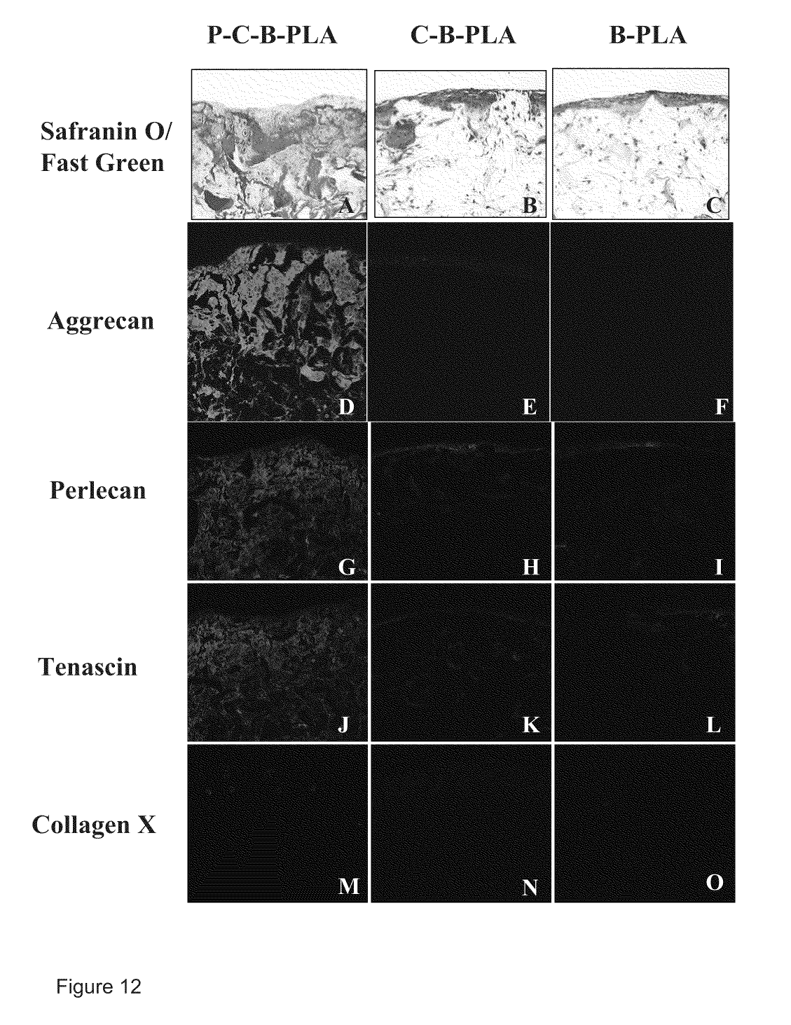

For experiments with primary mouse embryonic fibroblasts, cells seeded on P-C-BPLA scaffolds also demonstrated cartilage-like tissue formation after 21 days of culture (FIG. 12). Compared with cells cultured on C-B-PLA and B-PLA scaffolds, embryonic fibroblasts on P-C-B-PLA scaffolds displayed more GAG accumulation (as indexed by Alcian Blue staining; data not shown) and much higher chondrogenic marker expression (aggrecan, perlecan, tenascin). As with C3H10T1/2 cells, marginal expression of collagen X was observed on P-C-B scaffolds (FIG. 12M). In addition, embryonic fibroblasts cultured on P-C-B scaffolds deposited more extracellular matrix and displayed morphological characteristics more similar to chondrocytes than under the other conditions (FIG. 12, panels A-C).

### Discussion:

In developing biomimetic materials or scaffolds for tissue engineering, bioactive ECM molecules, such as collagen, fibronectin and laminin, have been used to improve biological activity of the scaffolds (51-53, 56). These components facilitate cell attachment, proliferation, differentiation, and the differentiated functions of cells (7, 34, 53, 56). Nonetheless, in the design strategies of biomimetic scaffolds, shorter polypeptide or peptide sequences often have advantages over the usually very large ECM proteins because of their superior bioavailability and stability properties and improved feasibility for mass production (53). In this regard, we have used a recombinant fragment of Pln, PlnDI, expressed by a transfected mammalian cell line and purified from conditioned medium. This fragment is substantially smaller than intact Pln (approximately 22 kDa versus 800 kDa core protein), is appropriately decorated with GAG chains, binds HBGFs well and promotes cell proliferation (7). Both intact Pln and PlnDI stimulate cartilage differentiation and promote the action of chondrogenic growth factors, such as BMP-2 and TGF-β1 (4, 6, 14, 19, 20). The BMP-2 plays key roles during chondrogenesis and was used to induce chondrogenic differentiation of mesenchymal stem cells and subsequent cartilage-like tissue formation in high-density culture (43-46). Thus, we considered that combining BMP-2 with proteins derived from cartilage ECM such as collagen type II and Pln would promote chondrogenic differentiation of mesenchymal stem cells. As a first step, we established that rhBMP-2 bound immobilized PlnDI with high affinity and stability and was abolished by digestion of HS chains in PlnDI with heparinases I, II and III. The latter observation demonstrated that the interaction between BMP-2 and PlnDI is dependent on the HS attached on its core protein. This interaction is consistent with previous studies demonstrating that HS can regulate and enhance BMP-2 functions (10, 11).

Collagen II is a fibril-forming collagen believed to be an effective substrate in engineering cartilage (32-36). Collagen II fibrils can interact with various proteoglycans that regulate collagen II fibril formation and ECM network assembly (25, 27-29). Heat denatured collagen II fibrils fail to interact with these proteoglycans (23, 27), suggesting that the triple helical structure of native collagen II is necessary for these interactions; however, the characteristics of the interactions are not very clear. Some studies indicate that proteoglycan binding to collagen II fibrils is mediated by CS or HS (23, 27-29), while other studies indicate a primary role for the proteoglycan core protein in these interactions (24-26). In the present studies, we demonstrated that the interaction between PlnDI and collagen II fibrils was dependent on both HS and CS. Moreover, the interaction was abolished by heat-denaturation of collagen II fibrils demonstrating a requirement for appropriate three-dimensional structure of the fibrils. The specificity of PlnDI binding to collagen II fibrils was verified by the demonstration of concentration dependent and saturable binding and competition by unlabeled PlnDI, but not the unrelated protein, BSA. In addition, PlnDI bound to collagen II fibrils much better than to collagen II monomers. Thus, it appears that the fibrillar configuration of collagen II contributes to optimal PlnDI binding.

These data indicate that P-C fibril complexes can be readily formed as a basis to develop new substrates for growth factor binding and cell culture. This substrate has superior functions than collagen II fibrils alone since it binds more HBGFs than collagen II-only substrates, regardless of the collagen II form or scaffold used. In addition, P-C fibril complexes not only retained but also sustained BMP-2 release, better than collagen II fibrils alone. To further investigate the potential value in tissue engineering, P-C fibril complexes were used to coat PLA scaffolds as described by Chen (51). rhBMP-2 interactions with different scaffolds were evaluated by a modified ELISA. These studies again demonstrated that P-C fibril complexes improved PLA scaffolds function via improved binding and retention of BMP-2.

To mimic events in chondrogenic differentiation (41-44) and chondrogenesis of mesenchymal cells in vitro, high density cell culture systems, including micromass or pellet cultures, have been used in combination with BMP-2 for both the C3H10T1/2 mesenchymal progenitor cell line (45, 46) as well as primary cultures of mouse embryonic fibroblasts and bone marrow stromal cells (43, 44). We also employed micromass cultures of C3H10T1/2 cells plated on different substrates on which rhBMP-2 was pre-loaded. Alcian blue staining showed that the micromass cultures plated on P-C-B fibrils appeared more differentiated, i.e., accumulated more GAG, than the micromass cultures on other substrates. To verify the differentiated state of C3H10T1/2 cells, expression of the chondrogenic marker genes, Sox9, aggrecan and collagen II, was evaluated by real time PCR. Consistent with the results of Alcian blue staining, mRNA expression of all chondrogenic markers was most robust when micromass cultures were plated on P-C-B fibrils. This effect is apparently due to the ability of P-C fibrils to bind and retain more BMP-2 than other substrates. Synthetic PLA scaffolds are easily processed into desired shapes, pore size and microstructure, and are mechanically strong, compared with collagen scaffolds (51).

Nonetheless, PLA scaffolds lack cell recognition signals, and their hydrophobic properties hinder uniform cell seeding in three dimensions (51, 53, 56). Therefore, synthetic scaffolds have been combined with bioactive molecules from ECM to improve their utility for tissue engineering. Surface modification of biomaterials with bioactive molecules is one method to make biomimetic materials and scaffolds (53). The finding that P-C fibril complexes effectively bind and retain BMP-2 suggested that these complexes are useful to coat and improve function of PLA scaffolds. Histological analysis reveal that cartilage-like tissue form in P-C-B-PLA with abundant GAG accumulation as shown by Safranin O-Fast Green staining. In contrast, there was no cartilage-like tissue in other scaffolds tested. Fibroblastic and adipocytelike cells mainly appeared in other scaffold constructs. The finding was confirmed by immunohistochemical analysis for expression of chondrogenic matrix components. Both aggrecan and Pln itself were used as additional markers of chondrogenesis (3-5, 12). During cartilage development, tenascin appears in mesenchymal cell condensations preceding chondrocyte differentiation while in adult cartilage, tenascin is abundantly expressed in articular cartilage and tracheal rings, but not mature bone matrix (16, 57, 58). Therefore, tenascin was used as another marker of chondrogenic differentiation. Cell lines may adapt or mutate during extended passaging in cell culture. Therefore, their responses are not necessarily reflective of responses of cells in tissues or primary cell cultures. To address this concern, primary cultures of mouse embryonic fibroblasts that possess stem cell qualities were utilized (43). Our results demonstrate that these primary cell cultures behaved similarly to the C3H10T1/2 cell line. This indicates that this approach can be used to generate cartilage-like tissue implants from primary cultures obtained from patients. Collectively, our observations indicate that scaffolds coated with P-C fibril complexes facilitate chondrogenic differentiation of mesenchymal progenitors in the presence of BMP-2 and are much superior to PLA scaffolds alone or coated with other combinations of PlnDI, collagen II fibrils and BMP-2. Chondrogenic differentiation of mesenchymal progenitor cells in vitro requires the complex influences of growth factors including BMPs and TGF-βs as well as cell-cell and cell-matrix interactions (41, 45, 50). In response to BMP-2, C3H10T1/2 cells undergo both chondrogenesis and osteogenesis (45). In our study, we observed little or no mineralized matrix under any condition tested. However, we detected modest alkaline phosphatase and collagen X expression in P-C-B-PLA scaffolds at regions closed to the exterior surface, demonstrating that some chondrocytes in this area underwent hypertrophic differentiation. Uniform chondrogenic differentiation is preferred for fabricating permanent cartilage. However, considering that the generation of a functional osteochondral junction is desirable for articular cartilage resurfacing, the finding that hypertrophic chondrocytes occur at the scaffold periphery is interesting and may even prove advantageous for proper tissue integration (54). These observations are in marked contrast to studies using PlnDI in combination with collagen type I and FGF-2 which effectively drives osteoblastic, rather than chondrocytic, differentiation (7). Therefore, both the growth factors used and the matrix components of the scaffold are strong influences on cell fate in tissue engineering applications.

In summary, by virtue of their ability to bind and retain key growth factors, PlnDI containing substrates are advantageous for biomimetic scaffolds for tissue regeneration, repair, and replacement. In addition to BMP-2, other HBGFs, such as FGF-2, VEGF, PDGF and HB-EGF, are believed to complex with PlnDI alone or when the PlnDI is coated onto scaffolds, either alone or in conjunction with an collagen type I or type II to promote chondrogenesis

FIG. 1. BMP-2 binding to PlnDI.

In rows A & B, PlnDI or PBS vehicle was immobilized on nitrocellulose. In row B, PlnDI or PBS also were digested with heparitinases I, II and III (HEPN) then immobilized on nitrocellulose as described in “Materials and Methods”. All wells subsequently were incubated with BMP-2 and bound BMP-2 detected. Panel C summarizes densitometric measurements performed on the dot blots above. From left to right in the bar graph: detection of binding of BMP-2 to PlnDI, PBS, PlnDI digested with HEPN and HEPN alone. Assays were performed in triplicate. Each bar indicates the mean±SD.

FIG. 2 PlnDI binding to collagen II fibrils.
A) Wells of 96-well microplates were coated with collagen II fibrils () or BSA (∘), followed by incubation with 100 μl of biotinylated PlnDI at the indicated concentrations as described in “Materials and Methods”. B) Non biotinylated PlnDI was used to compete for biotinylated PlnDI binding to collagen II fibrils at increasing molar ratios of PlnDI/biotinylated PlnDI as described in “Materials and Methods”. Assays were performed in triplicate. Values given are the mean±SD in each case.
FIG. 3 PlnDI binding to collagen II fibrils is HS and CS dependent.
A) Binding of biotinylated PlnDI, digested or undigested with heparinases I, II and III (HEPN) and/or chondroitinase ABC (CHON) as indicated on the figure, to collagen II fibrils coated on polyethylene wells was determined as described in “Materials and Methods”. B) Biotinylated PlnDI was mixed with either HS (250 μg/ml) or CS (250 μg/ml), and then incubated with collagen II fibrils coated on polyethylene wells. Binding was determined as described in “Materials and Methods”. All assays were performed in triplicate and results of a representative experiment are shown. Each bar indicates the mean±SD.
FIG. 4 PlnDI binding to different forms of collagen II.

Binding of biotinylated PlnDI to collagen II fibrils, collagen II monomers, heat-denatured collagen II fibrils or BSA was determined as described in “Materials and Methods”. Each assay was performed in triplicate and the results of a representative experiment are shown. Each bar represents the mean±SD.

FIG. 5 BMP-2 binding to PlnDI associated with collagen II fibrils.

Binding of BMP-2 to PlnDI/collagen II fibril complexes (P-C), heparitinase-digested PlnDI digested/collagen II fibril complexes (DH-P-C), chondroitinase ABC-digested PlnDI/collagen II fibril complexes (DC-P-C), was evaluated with a solid phase binding assay as described in “Materials and Methods”. Coating with collagen II alone (Coll-II) or BSA were used as controls. Each bar indicates the mean±SD of triplicate determinations from a representative experiment.

FIG. 6 BMP-2 release from PlnDI/collagen II fibril complexes and collagen II fibrils.

Complexes of PlnDI/collagen II fibrils (PlnDI/Coll-II) () and collagen II fibrils alone (Coll-II) (∘) were pre-coated on surfaces and subsequently incubated with BMP-2. Released BMP-2 was determined as the indicated time by ELISA as described in “Materials and Methods”. A) The amount of BMP-2 bound was calculated by subtracting the amount of BMP-2 released from the amount determined to be bound at time zero. B) BMP-2 release was calculated as the percentage of BMP-2 released at the indicated time relative to the amount bound to the scaffold at time zero. All points reflect the means±SD of triplicate determinations in each case.

FIG. 7 Alcian Blue staining of micromass cultures of C3HT1/2 cells on different substrates.

High density micromass cultures of C3H10T1/2 cells (1×10₅/10 ul) were plated on the indicated substrates for 6 days followed by Alcian Blue staining as described in “Materials and Methods”. The substrates used were PlnDI-collagen II fibril-BMP2 complexes (P-C-B), collagen II fibril-BMP-2 complexes (C-B), PlnDI-collagen II fibril complexes (P-C) and collagen II fibrils alone (C).

FIG. 8 Chondrogenic differentiation marker mRNA expression by micromass cultures of C3H10T1/2 cells.

Total RNA was extracted from micromass cultures of C3H10T1/2 cells cultured on different substrates (abbreviations same as described in legend to panel 7) after 6 days of culture and relative levels of expression of mRNA encoding collagen II (A), aggrecan (B) or sox9 (C) was evaluated by real-time RT-PCR as described in “Materials and Methods”. Values represent means±SD of triplicate determinations of separate RNA isolates in each case.

FIG. 9 BMP-2 binding to different three-dimensional scaffolds.

An ELISA-based assay was used to determine BMP-2 binding to scaffolds as described in “Materials and Methods”. The scaffolds were constructed of PlnDI/collagen II fibrils-PLA scaffolds (PlnDI/Coll-II-PLA), collagen II fibrils-PLA (Coll-II-PLA) or PLA alone (PLA). The upper panel shows a photograph of scaffolds retaining the blue reaction product generated by the ELISA indicating BMP-2 retention. The lower bar graph shows the quantitation of dye in each scaffold following extraction and measurement of OD₄₅₀ in the extracts as described in “Materials and Methods”. Each bar represents the mean±SD of triplicate determinations.

FIG. 10 Histological analysis of C3H10T1/2 cells seeded in different scaffolds.

C3H10T1/2 cells were seeded and cultured dynamically for 21 days on each scaffold followed by Safranin O-fast green straining (A-F), alkaline phosphatase staining (pink, G-I) or von Kossa staining (J-L) as described in “Materials and Methods”. The scaffolds were constructed of PlnDI-collagen II fibrils PLA (A), collagen II fibrils-PLA (B) or PLA (C), BMP-2-PlnDI-collagen II-PLA (D, G, J), BMP-2-collagen II fibrils-PLA (E, H, K) or BMP-2-PLA (F, I, L). (Scale bar=200 μm).

FIG. 11 Immunohistochemical analysis of chondrogenic markers by C3H101/2 cells seeded in different scaffolds.

C3H10T1/2 cells-scaffolds were seeded and cultured dynamically for 21 days in different scaffolds as described in “Materials and Methods”. The scaffolds used were PlnDIcollagen II fibrils-BMP-2-PLA (P-C-B-PLA, panels A, D, G and J), collagen II fibrils-BMP-2-PLA (C-B-PLA, panels B, E, H and K) and BMP-2-PLA (B-PLA, panels C, F, I and K). The sections of cell-scaffold constructs were stained for aggrecan (A-C), perlecan (D-F), tenascin (G-I) or collagen X (J-K). (Scale bar=200 μm). The absence of staining contrast in the C-B-PLA and B-PLA is indicative of a lack of regenerated cartilage tissue.

FIG. 12 Histological and Immunohistochemical analysis of chondrogenic markers by mouse embryonic fibroblasts (MEFs) seeded in different scaffolds.

MEFs were seeded and cultured dynamically for 21 days in the indicated scaffolds followed by sectioning and staining by Safranin O-fast green (A-C) or immunostaining for aggrecan (D-F), perlecan (domain IV) (G-I), tenascin (J-L) and collagen X (M-0) as described in “Materials and Methods”. The abbreviation for the scaffolds in each column are the same as described in the legend to FIG. 11. (Scale bar =200 μm). The absence of staining contrast in the C-B-PLA and B-PLA is indicative of a lack of regenerated cartilage tissue.

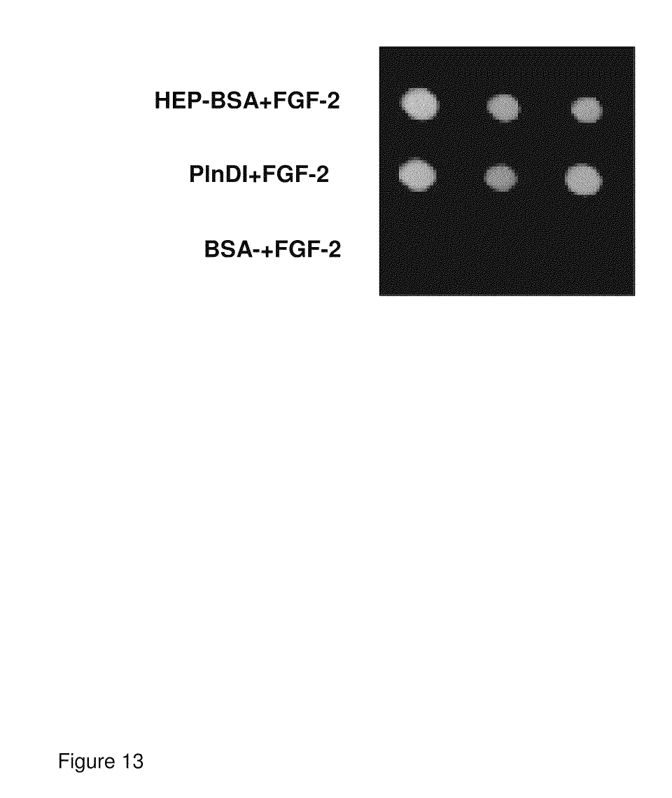

FIG. 13 PlnDI binding of FGF-2 vs. HEP-BSA binding of FGF-2

The blots illustrate Heparin-BSA complex and PlnDI complexes binding bFGF when attached to nitrocellulose membrane (“dot blot assay”). The PlnDI concentration used on the membrane was 0.1 mg/well (middle row), whereas the heparin-BSA complex had to be applied at a 3.0 mg/well concentration (upper row) to get similar signal. A BSA control was used which showed no binding at all with FGF-2.

TABLE I
Specific primers used for real time PCR

| Genes | Forward | Reverse |
|---|---|---|
| β- | 5′AAATCGTGCGTGACATCAAAGA3′ | 5′GCCATCTCCTGCTCGAAGTC3′ |
| actin | | |
| Collagen | 5′CTCATCCAGGGCTCCAATGA3′ | |
| II | 5′TCCTTCAGGGCAGTGTATGTGA3′ | |
| Aggrecan | 5′CAGGGTTCCCAGTGTTCAGT3′ | 5′CCAGAAGACTCTCCACTGCC3′ |
| Sox9 | 5′GAGGCCACGGAACAGACTCA3′ | |
| | 5′CAGCGCCTTGAAGATAGCATT3′ | |

While various embodiments of the present invention are presented above, it is noted that these foregoing examples are provided merely for purposes of explanation and are not for purposes of limitation. While the present invention may be described with reference to an exemplary embodiment, the language used to set forth the exemplary embodiment are words of description and not words of limitation. Although the present invention is described with reference to particular means, materials and structures, the present invention is not intended to be limited to the particulars disclosed, rather the present invention extends to all present and later developed equivalents of those set forth herein as appreciated by one of ordinary skill in the relevant art. All references, including U.S. patents and patent applications, cited herein are hereby incorporated by reference herein in their entireties.

### REFERENCES

1. Poole, A. R., Kojima, T., Yasuda, T., Mwale, F., Kobayashi, M. and Layerty, S. Composition and structure of articular cartilage: a template for tissue repair. Clin. Orthop. Relat. Res., S26, 2001.
2. Knudson, C. B. and Knudson, W. Cartilage proteoglycans. Semin Cell Dev Biol., 12, 69, 2001.
3. Gustafsson, E., Aszodi, A., Ortega, N., Hunziker, E. B., Denker, H. W., Werb, Z. and Fassler, R. Role of collagen type II and perlecan in skeletal development. Ann N Y Acad Sci, 995, 140, 2003.
4. Gomes, R. R., Jr., Farach-Carson, M. C. and Carson, D. D. Perlecan functions in chondrogenesis: insights from in vitro and in vivo models. Cells Tissues Organs, 176, 79, 2004.
5. Murdoch, A. D., Dodge, G. R., Cohen, I., Tuan, R. S. and Iozzo, R. V. Primary structure of the human heparan sulfate proteoglycan from basement membrane (HSPG2/perlecan). A chimeric molecule with multiple domains homologous to the low density lipoprotein receptor, laminin, neural cell adhesion molecules, and epidermal growth factor. J Biol Chem, 267, 8544, 1992.
6. Kim-Safran, C. B., Gomes, R. R., Brown, A. J. and Carson, D. D. Heparan sulfate proteoglycans: coordinators of multiple signaling pathways during chondrogenesis. Birth Defects Res C Embryo Today, 72, 69, 2004.
7. Yang, W. D., Gomes, R. R., Jr., Alicknavitch, M., Farach-Carson, M. C. and Carson, D. D. Perlecan domain I promotes fibroblast growth factor 2 delivery in collagen I fibril scaffolds. Tissue Eng, 11, 76, 2005.
8. Knox, S., Merry, C., Stringer, S., Melrose, J. and Whitelock, J. Not all perlecans are created equal: interactions with fibroblast growth factor (FGF) 2 and FGF receptors. J Biol Chem, 277, 14657, 2002.
9. Jiang, X. and Couchman, J. R. Perlecan and tumor angiogenesis. J Histochem Cytochem, 51, 1393, 2003.
10. Ruppert, R., Hoffmann, E. and Sebald, W. Human bone morphogenetic protein 2 contains a heparin-binding site which modifies its biological activity. Eur J Biochem, 237, 295, 1996.
11. Takada, T., Katagiri, T., Ifuku, M., Morimura, N., Kobayashi, M., Hasegawa, K., Ogamo, A. and Kamijo, R. Sulfated polysaccharides enhance the biological activities of bone morphogenetic proteins. J Biol Chem, 278, 43229, 2003.
12. Arikawa-Hirasawa, E., Watanabe, H., Takami, H., Hassell, J. R. and Yamada, Y. Perlecan is essential for cartilage and cephalic development. Nat Genet, 23, 354, 1999.
13. Kosir, M. A., Quinn, C. C., Wang, W. and Tromp, G. Matrix glycosaminoglycans in the growth phase of fibroblasts: more of the story in wound healing. J Surg Res, 92, 45, 2000.
14. French, M. M., Smith, S. E., Akanbi, K., Sanford, T., Hecht, J., Farach-Carson, M. C. and Carson, D. D. Expression of the heparan sulfate proteoglycan, perlecan, during mouse embryogenesis and perlecan chondrogenic activity in vitro. J Cell Biol, 145, 1103, 1999.
15. Handler, M., Yurchenco, P. D. and Iozzo, R. V. Developmental expression of perlecan during murine embryogenesis. Dev Dyn, 210, 130, 1997.
16. Aszodi, A., Bateman, J. F., Gustafsson, E., Boot-Handford, R. and Fassler, R. Mammalian skeletogenesis and extracellular matrix: what can we learn from knockout mice? Cell Struct Funct, 25, 73, 2000.
17. Costell, M., Gustafsson, E., Aszodi, A., Morgelin, M., Bloch, W., Hunziker, E., Addicks, K., Timpl, R. and Fassler, R. Perlecan maintains the integrity of cartilage and some basement membranes. J Cell Biol, 147, 1109, 1999.
18. Hassell, J., Yamada, Y. and Arikawa-Hirasawa, E. Role of perlecan in skeletal development and diseases. Glycoconj J, 19, 263, 2002.
19. French, M. M., Gomes, R. R., Jr., Timpl, R., Hook, M., Czymmek, K., Farach-Carson, M. C. and Carson, D. D. Chondrogenic activity of the heparan sulfate proteoglycan perlecan maps to the N-terminal domain I. J Bone Miner Res, 17, 48, 2002.
20. Gomes, R. R., Jr., Farach Carson, M. C. and Carson, D. D. Perlecan-stimulated nodules undergo chondrogenic maturation in response to rhBMP-2 treatment in vitro. Connect Tissue Res, 44 Suppl 1, 196, 2003.
21. Miller, S. A., Brown, A. J., Farach-Carson, M. C. and Kirn-Safran, C. B. HIP/RPL29 down-regulation accompanies terminal chondrocyte differentiation. Differentiation, 71, 322, 2003.
22. Paine-Saunders, S., Viviano, B. L., Economides, A. N. and Saunders, S. Heparan sulfate proteoglycans retain Noggin at the cell surface: a potential mechanism for shaping bone morphogenetic protein gradients. J Biol Chem, 277, 2089, 2002.
23. Hedbom, E. and Heinegard, D. Binding of fibromodulin and decorin to separate sites on fibrillar collagens. J Biol Chem, 268, 27307, 1993.
24. Oldberg, A., Antonsson, P., Lindblom, K. and Heinegard, D. A collagen-binding 59-kd protein (fibromodulin) is structurally related to the small interstitial proteoglycans PG-S1 and PG-S2 (decorin). Embo J, 8, 2601, 1989.
25. Vynios, D. H., Papageorgakopoulou, N., Sazakli, H. and Tsiganos, C. P. The interactions of cartilage proteoglycans with collagens are determined by their structures. Biochimie, 83, 899, 2001.
26. Hedlund, H., Hedbom, E., Heineg rd, D., Mengarelli-Widholm, S., Reinholt, F. P. and Svensson, O. Association of the aggrecan keratan sulfate-rich region with collagen in bovine articular cartilage. J Biol Chem, 274, 5777, 1999.
27. Smith, G. N., Jr., Williams, J. M. and Brandt, K. D. Interaction of proteoglycans with the pericellular (1 alpha, 2 alpha, 3 alpha) collagens of cartilage. J Biol Chem, 260, 10761, 1985.
28. San Antonio, J. D., Karnovsky, M. J., Gay, S., Sanderson, R. D. and Lander, A. D. Interactions of syndecan-1 and heparin with human collagens. Glycobiology, 4, 327, 1994.
29. Munakata, H., Takagaki, K., Majima, M. and Endo, M. Interaction between collagens and glycosaminoglycans investigated using a surface plasmon resonance biosensor. Glycobiology, 9, 1023, 1999.
30. Zhu, Y., Oganesian, A., Keene, D. R. and Sandell, L. J. Type IIA procollagen containing the cysteine-rich amino propeptide is deposited in the extracellular matrix of prechondrogenic tissue and binds to TGF-beta1 and BMP-2. J Cell Biol, 144, 1069, 1999.
31. Sieron, A. L., Louneva, N. and Fertala, A. Site-specific interaction of bone morphogenetic protein 2 with procollagen II. Cytokine, 18, 214, 2002.
32. Fertala, A., Han, W. B. and Ko, F. K. Mapping critical sites in collagen II for rational design of gene-engineered proteins for cell-supporting materials. J Biomed Mater Res, 57, 48, 2001.
33. Nehrer, S., Breinan, H. A., Ramappa, A., Shortkroff, S., Young, G., Minas, T., Sledge, C. B., Yannas, I. V. and Spector, M. Canine chondrocytes seeded in type I and type II collagen implants investigated in vitro. J Biomed Mater Res, 38, 95, 1997.
34. Veilleux, N. H., Yannas, I. V. and Spector, M. Effect of passage number and collagen type on the proliferative, biosynthetic, and contractile activity of adult canine articular chondrocytes in type I and II collagen-glycosaminoglycan matrices in vitro. Tissue Eng, 10, 119, 2004.
35. Hsu, S. H., Tsai, C. L. and Tang, C. M. Evaluation of cellular affinity and compatibility to biodegradable polyesters and Type-II collagen-modified scaffolds using immortalized rat chondrocytes. Artif Organs, 26, 647, 2002.
36. Taguchi, T., Ikoma, T. and Tanaka, J. An improved method to prepare hyaluronic acid and type II collagen composite matrices. J Biomed Mater Res, 61, 330, 2002.
37. Pieper, J. S., van der Kraan, P. M., Hafmans, T., Kamp, J., Buma, P., van Susante, J. L., van den Berg, W. B., Veerkamp, J. H. and van Kuppevelt, T. H. Crosslinked type II collagen matrices: preparation, characterization, and potential for cartilage engineering. Biomaterials, 23, 3183, 2002.
38. Lee, V., Cao, L., Zhang, Y., Kiani, C., Adams, M. E. and Yang, B. B. The roles of matrix molecules in mediating chondrocyte aggregation, attachment, and spreading. J Cell Biochem, 79, 322, 2000.
39. Buma, P., Pieper, J. S., van Tienen, T., van Susante, J. L., van der Kraan, P. M., Veerkamp, J. H., van den Berg, W. B., Veth, R. P. and van Kuppevelt, T. H. Cross-linked type I and type II collagenous matrices for the repair of full-thickness articular cartilage defects—a study in rabbits. Biomaterials, 24, 3255, 2003.
40. Mueller, S. M., Shortkroff, S., Schneider, T. O., Breinan, H. A., Yannas, I. V. and Spector, M. Meniscus cells seeded in type I and type II collagen-GAG matrices in vitro. Biomaterials, 20, 701, 1999.
41. Hall, B. K. and Miyake, T. All for one and one for all: condensations and the initiation of skeletal development. Bioassays, 22, 138, 2000.
42. Tuan, R. S. Biology of developmental and regenerative skeletogenesis. Clin Orthop Relat. Res, S105, 2004.
43. Lengner, C. J., Lepper, C., van Wijnen, A. J., Stein, J. L., Stein, G. S. and Lian, J. B. Primary mouse embryonic fibroblasts: a model of mesenchymal cartilage formation. J Cell Physiol, 200, 327, 2004.
44. Schmitt, B., Ringe, J., Haupl, T., Notter, M., Manz, R., Burmester, G. R., Sittinger, M. and Kaps, C. BMP2 initiates chondrogenic lineage development of adult human mesenchymal stem cells in high-density culture. Differentiation, 71, 567, 2003.
45. Shea, C. M., Edgar, C. M., Einhom, T. A. and Gerstenfeld, L. C. BMP treatment of C3H10T1/2 mesenchymal stem cells induces both chondrogenesis and osteogenesis. J Cell Biochem, 90, 1112, 2003.
46. Seghatoleslami, M. R. and Tuan, R. S. Cell density dependent regulation of AP-1 activity is important for chondrogenic differentiation of C3H10T1/2 mesenchymal cells. J Cell Biochem, 84, 237, 2002.
47. Hanada, K., Solchaga, L. A., Caplan, A. I., Hering, T. M., Goldberg, V. M., Yoo, J. U. and Johnstone, B. BMP-2 induction and TGF-beta 1 modulation of rat periosteal cell chondrogenesis. J Cell Biochem, 81, 284, 2001.
48. Izzo, M. W., Pucci, B., Tuan, R. S. and Hall, D. J. Gene expression profiling following BMP-2 induction of mesenchymal chondrogenesis in vitro. Osteoarthritis Cartilage, 10, 23, 2002.
49. Atkinson, B. L., Fantle, K. S., Benedict, J. J., Huffer, W. E. and Gutierrez-Hartmann, A. Combination of osteoinductive bone proteins differentiates mesenchymal C3H/10T1/2 cells specifically to the cartilage lineage. J Cell Biochem, 65, 325, 1997.
50. Chen, G., Ushida, T. and Tateishi, T. Poly(DL-lactic-co-glycolic acid) sponge hybridized with collagen microsponges and deposited apatite particulates. J Biomed Mater Res, 57, 8, 2001.
51. Unsworth, J. M., Rose, F. R., Wright, E., Scotchford, C. A. and Shakesheff, K. M. Seeding cells into needled felt scaffolds for tissue engineering applications. J Biomed Mater Res A, 66, 425, 2003.
52. Shin, H., Jo, S. and Mikos, A. G. Biomimetic materials for tissue engineering. Biomaterials, 24, 4353, 2003.
53. Noth, U., Tuli, R., Osyczka, A. M., Danielson, K. G. and Tuan, R. S. In vitro engineered cartilage constructs produced by press-coating biodegradable polymer with human mesenchymal stem cells. Tissue Eng, 8, 131, 2002.
54. Yang, W. D., Chen, S. J., Mao, T. Q., Chen, F. L., Lei, D. L., Tao, K., Tang, L. H. and Xiao, M. G. A study of injectable tissue-engineered autologous cartilage. Chin J Dent Res, 3, 10, 2000.
55. Jones, F. S. and Jones, P. L. The tenascin family of ECM glycoproteins: structure, function, and regulation during embryonic development and tissue remodeling. Dev Dyn, 218, 235, 2000.
56. Qi, W. N. and Scully, S. P. Type II collagen modulates the composition of extracellular matrix synthesized by articular chondrocytes. J Orthop Res, 21, 282, 2003.
57. Yamaguchi, N. and Kiick, Kristi L. Polysaccharide-Poly(ethylene glycol) Star Copoloymer as a Scaffold for the Production of Bioactive Hydrogels. Biomacromolecules, 6 (4) 1921-30, 2005.

1. A biomaterial having immobilized thereon a proteoglycan-growth factor complex comprising (1) a proteoglycan that comprises an amino acid sequence of the core protein of domain I of a mammalian perlecan or that comprises an amino acid sequence having at least 90% homology to the core protein of domain I of a mammalian perlecan to which proteoglycan at least one glycosaminoglycan is attached and (2) at least one growth factor, said immobilized proteoglycan-growth factor complex being present in the biomaterial in a sufficient amount for sustained release of a therapeutically effective dose of growth factor to repair and regenerate tissue at a wound site over a predetermined period of time.

2. The biomaterial of claim 1 wherein the proteoglycan-growth factor complex releases less than 25% of the growth factor over a predetermined period of three days.

3. The biomaterial of claim 1 wherein the proteoglycan-growth factor complex releases 3 to 12% of the growth factor over a predetermined period of three days.

4. The biomaterial of claim 1 wherein the proteoglycan-growth factor complex releases less than 60% of the growth factor over a predetermined period of twelve days.

5. The biomaterial of claim 1 wherein the proteoglycan-growth factor complex releases 30 to 50% of the growth factor over a predetermined period of twelve days.

6. The biomaterial of claim 1 wherein the proteoglycan is bound to collagen.

7. The biomaterial of claim 6 wherein the collagen is a collagen fibril selected from the group consisting of collagen types I-XIII and pro-collagen.

8. The biomaterial of claim 1 wherein the at least one growth factor is selected from the group consisting of TGFB, FGF-2, BMP-2, VEGF, PDGF and HB-EGF.

9. The biomaterial of claim 8 wherein the growth factor is BMP-2.

10. The biomaterial of claim 1 wherein the proteoglycan has a molecular size of less than 100 kDa.

11. The biomaterial of claim 1 wherein the proteoglycan has an amino acid sequence of a mammalian perlecan domain I to which conservative amino acid substitutions have been made.

12. The biomaterial of claim 1 wherein the amino acid sequence of the proteoglycan comprises a sequence having at least 90% homology to SEQ ID NO:1.

13. A scaffold or a hydrogel comprising the biomaterial of claim 1.

14. A pharmaceutical composition for injection comprising the biomaterial of claim 1 and a pharmaceutically acceptable adjuvant.

15. A method of treating or preventing cartilage damage at a wound site in a mammal by sustained release of growth factor comprising introducing at the wound site the biomaterial of claim 1.

16. The method of claim 15 wherein the proteoglycan-growth factor complex releases less than 25% of the growth factor over a predetermined period of three days.

17. The method of claim 15 wherein the proteoglycan-growth factor complex releases 3 to 12% of the growth factor over a predetermined period of three days.

18. The method of claim 15 wherein the proteoglycan-growth factor complex releases less than 60% of the growth factor over a predetermined period of twelve days.

19. The method of claim 15 wherein the proteoglycan-growth factor complex releases 30 to 50% of the growth factor over a predetermined period of twelve days.

20. The method of claim 15 wherein the proteoglycan-growth factor is bound to collagen.

21. The method of claim 15 wherein the at least one growth factor associated with the proteoglycan comprises a heparin-binding growth factor.

22. The method of claim 21 wherein the heparin-binding growth factor is BMP-2.

23. The method of claim 15, wherein the proteoglycan comprises domain I of perlecan.

24. The method of claim 15 wherein the proteoglycan has a molecular size of less than 100 kDa.

25. The method of claim 15 wherein the mammal suffers from osteoarthritis and the biomaterial is administered directly to a joint afflicted with osteoarthritis.

26. A therapeutic composition comprising a diluent and a proteoglycan-growth factor complex, said complex comprising (1) a proteoglycan that comprises an amino acid sequence of the core protein of domain I of a mammalian perlecan or that comprises an amino acid sequence having at least 90% homology to the core protein of domain I of a mammalian perlecan to which proteoglycan at least one glycosaminoglycan is attached and (2) at least one growth factor, said immobilized proteoglycan-growth factor complex being present in the composition in a sufficient amount for sustained release of a therapeutically effective dose of growth factor to repair and regenerate tissue at a wound site over a predetermined period of time.

27. The therapeutic composition of claim 26 wherein the proteoglycan-growth factor complex releases less than 25% of the growth factor over a predetermined period of three days.

28. The therapeutic composition of claim 26 wherein the proteoglycan-growth factor complex releases 3 to 12% of the growth factor over a predetermined period of three days.

29. The therapeutic composition of claim 26 wherein the proteoglycan-growth factor complex releases less than 60% of the growth factor over a predetermined period of twelve days.

30. The therapeutic composition of claim 26 wherein the proteoglycan-growth factor complex releases 30 to 50% of the growth factor over a predetermined period of twelve days.

31. The therapeutic composition of claim 26 wherein the growth factor is BMP-2.

32. The therapeutic composition of claim 26 wherein the proteoglycan has a molecular size of less than 100 kDa.

33. A method of treating or preventing cartilage damage at a wound site in a mammal by sustained release of growth factor comprising introducing at the wound site the therapeutic composition of claim 26.

34. The method of claim 33 wherein the proteoglycan-growth factor complex releases less than 25% of the growth factor over a predetermined period of three days.

35. The method of claim 33 wherein the proteoglycan-growth factor complex releases 3 to 12% of the growth factor over a predetermined period of three days.

36. The method of claim 33 wherein the proteoglycan-growth factor complex releases less than 60% of the growth factor over a predetermined period of twelve days.


SEQUENCE LISTING (10 sequences)

SEQ ID NO: 1 - protein, 172 aa, Human
VTHGLRAYDGLSLPEDIETVTASQMRWTHSYLSDDEYMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVP

SEQ ID NO: 2 - protein, 173 aa, Mouse
VTHGLRAYDGLSLPEDTETVTASRYGWTYSYLSDDEDLLADDASGDGLGSGDVGSGDFQMVYFRALVNFTRSIEYSPQLEDASAKEFREVSEAVVEKLEPEYRKIPGDQIVSVVFIKELDGWVFVELDVGSEGNADGSQIQEVLHTVVSSGSIGPYVTSPWGFKFRRLGTVPQ

SEQ ID NO: 3 - DNA, 22 bp, Artificial Sequence
  Synthetic
aaatcgtgcgtgacatcaaaga

SEQ ID NO: 4 - DNA, 20 bp, Artificial Sequence
  Synthetic
gccatctcctgctcgaagtc

SEQ ID NO: 5 - DNA, 20 bp, Artificial Sequence
  Synthetic
ctcatccagggctccaatga

SEQ ID NO: 6 - DNA, 22 bp, Artificial Sequence
  Synthetic
tccttcagggcagtgtatgtga

SEQ ID NO: 7 - DNA, 20 bp, Artificial Sequence
  Synthetic
cagggttcccagtgttcagt

SEQ ID NO: 8 - DNA, 20 bp, Artificial Sequence
  Synthetic
ccagaagactctccactgcc

SEQ ID NO: 9 - DNA, 20 bp, Artificial Sequence
  Synthetic
gaggccacggaacagactca

SEQ ID NO: 10 - DNA, 21 bp, Artificial Sequence
  Synthetic
cagcgccttgaagatagcatt